(19) United States
(12) Patent Application Publication
(10) Pub. No.: US 2023/0113477 A1
(43) Pub. Date: Apr. 13, 2023

(54) SOFT TISSUE-HARD TISSUE INTERFACE FIXATION DEVICE

(71) Applicants: Washington University, St. Louis, MO (US); Columbia University, New York, NY (US)

(72) Inventors: Guy Genin, St. Louis, MO (US); Ethan Hoppe, St. Louis, MO (US); Dong Hwan Yoon, St. Louis, MO (US); Stavros Thomopoulos, New York, NY (US); Iden Kurtaliaj, New York, NY (US); Liana Tedesco, New York, NY (US); David Kovacevic, New York, NY (US); Victor Birman, St. Louis, MO (US); Lester Smith, Indianapolis, IN (US); Leesa Galatz, St. Louis, MO (US); William Levine, New York, NY (US)

(73) Assignees: Washington University, St. Louis, MO (US); Columbia University, New York, NY (US)

(21) Appl. No.: 17/932,232

(22) Filed: Sep. 14, 2022

Related U.S. Application Data
(63) Continuation-in-part of application No. 17/766,503, filed on Jan. 1, 0001.
(60) Provisional application No. 63/244,210, filed on Sep. 14, 2021.
(60) Provisional application No. 63/040,096, filed on Jun. 17, 2020.
(60) Provisional application No. 62/910,273, filed on Oct. 3, 2019.

(51) Int. Cl. A61B 17/04

(52) U.S. Cl. CPC A61B 17/0401; A61B 2017/0412; A61B 2017/0414

(57) ABSTRACT

Devices and methods for joining a first and second tissue in a patient are disclosed that include a base with opposed first and second surfaces, and a plurality of recurved tines oriented to a tine axis and extending from the first surface of the base. The base defines four suture holes extending through the first and second surfaces of the base and configured to receive at least one suture passing between the first and second surfaces of the base. The plurality of recurved tines provides unidirectional traction of the first tissue along the tine axis toward the first surface to secure the device to the first tissue. The device is secured to the second tissue at the second surface with the at least one suture secured to at least one anchor secured to the second tissue.

### CROSS-REFERENCE TO RELATED APPLICATIONS

This application is a continuation-in-part of U.S. Non-Provisional Patent Application Ser. No. 17/766,503 filed Apr. 4, 2022, which is incorporated herein by reference in its entirety. U.S. Non-Provisional Patent Application Ser. No. 17/766,503 is the U.S. national phase of international Application No. PCT/US20/54320 filed Oct. 5, 2020, This application claims priority from U.S. Provisional Application Ser. No. 62/910,273 filed on Oct. 3, 2019, which is incorporated herein by reference in its entirety. This application also claims priority from U.S. Provisional Application Ser. No. 63/040,096 filed on Jun. 17, 2020, which is incorporated herein by reference in its entirety. This application also claims priority from U.S. Provisional Application Ser. No. 63/244,210 filed on Sep. 14, 2021, which is incorporated herein by reference in its entirety.

### STATEMENT REGARDING FEDERALLY SPONSORED RESEARCH OR DEVELOPMENT

This invention was made with government support under EB016422 awarded by the National Institutes of Health and by CMMI1548571 awarded by the National Science Foundation. The government has certain rights in the invention.

### MATERIAL INCORPORATED-BY-REFERENCE

Not applicable.

### FIELD OF THE DISCLOSURE

The present disclosure generally relates to devices and methods to affix a soft tissue to a hard tissue, such as tendon-to-bone and ligament-to-bone attachments.

### BACKGROUND OF THE DISCLOSURE

Repair of torn tendon-to-bone and ligament-to-bone attachments is a perennial challenge in orthopedic surgery. Some soft tissue repairs have very high failure rates. A large body of literature illustrates the high anatomical failure rate after rotator cuff repair. For example, 20-94% of rotator cuff repairs result in the recurrence of tears. Multiple factors affect rotator cuff healing, including patient-related factors, as well as surgical factors. Increased strength of initial repair has been documented to improve healing rates; however, current methods only utilize sutures and suture anchors. A device is needed that will provide effective fixation of tendon/ligament to bone during post-surgical healing while offloading high stresses and allowing for re-integration of tendon/ligament to bone. Such a device is applicable to many different anatomical sites in orthopedics, as well as in other surgical subspecialties.

Current technology for this surgery involves suturing the tendon to bone. Suture techniques often result in gap formation or rupture at the interface, leading to with poor healing. An advantage of the disclosed device is that it provides more uniform contact between tendon and bone by augmenting or replacing sutures with a field of attachment points.

There are hundreds of thousands of musculoskeletal tissue surgical repairs in the United States yearly. Poor soft tissue-hard tissue fixation after surgical repair, however, remains a clinical challenge since a strong and tough attachment is not currently achieved using existing approaches.

Other objects and features will be in part apparent and in part pointed out hereinafter.

### SUMMARY OF THE DISCLOSURE

In one aspect, a device for joining a first tissue to a second tissue in a patient in need, the device comprising: a base comprising opposed first and second surfaces, the base defining four suture holes extending through the first and second surfaces of the base, each suture hole configured to receive at least one suture passing between the first and second surfaces; and a plurality of recurved tines oriented to a tine axis and extending from the first surface of the base, the plurality of recurved tines providing unidirectional traction of the first tissue along the tine axis toward the first surface; wherein the first tissue is secured to the first surface of the device at the plurality of recurved tines and the second tissue is secured to the device at the second surface to join the first tissue to the second tissue. In some aspects, the at least one suture is configured to secure to at least one suture anchor, the at least one suture anchor secured to the second tissue. In some aspects, at least a portion of the suture holes of the base are configured to receive anchor screws, and the device is secured to the second tissue using a combination of anchor screws and sutures. In some aspects, the first tissue and second tissue are selected independently from a hard tissue and a soft tissue. In some aspects, the hard tissue comprises a bone tissue. In some aspects, the soft tissue is a soft connective tissue selected from a ligament, a tendon, a muscle, a cartilage tissue, a meniscus, a fibrocartilage tissue, an adipose tissue, and any combination thereof In some aspects, the first tine axis is oriented in a direction opposite to the second tine axis. In some aspects, the base comprises essentially planar first and second surfaces. In some aspects, the base further comprises a flexible material wherein the base is further configured to conform to match a contour of the second tissue, to fold or roll to fit through an arthroscopic cannula, and any combination thereof. In some aspects, the base further defines a plurality of pores, each pore of the plurality of pores extending at least partly from the first surface to the second surface. In some aspects, at least a portion of the plurality of pores are arranged in a distribution pattern over at least a portion of the base, the pattern selected from one of a random distribution, a uniform distribution, a lattice distribution, and any combination thereof. In some aspects, the lattice distribution is selected from one of a row and column distribution, a cubic lattice distribution, and a hexagonal lattice distribution. In some aspects, the distribution pattern of the pores is configured to reduce or equalize stresses in the plurality of recurved tines, to equalize mechanical loadings in the plurality of recurved tines, to reduce maximum stress in the base, reduce a likelihood of failure of the base, and any combination thereof. In some aspects, at least one of the first surface and the second surface are contoured, the first surface contoured to conform to a surface of the first tissue, and the second surface contoured to conform to a surface of the second tissue. In some aspects, at least one of the first surface and the second surface are contoured, the first surface contoured to conform to a surface of the first tissue of an individual patient, and the second surface contoured to conform to a surface of the second tissue of the individual patient. In some aspects, the base further comprises a length ranging from about 5 mm to about 18 mm and a width ranging from about 3 mm to about 9 mm. In some aspects, the base comprises a length of about 17.5 mm and a width of about 8 mm. In some aspects, a portion of the plurality of recurved tines vary in a tine characteristic, the tine characteristic comprising a tine size, a tine spacing, a tine orientation different from the tine axis, or any combination thereof; and each tine characteristic of each tine from the portion of the plurality of recurved tines is selected to reduce or equalize stresses in the tines, reduce stresses in the attached first tissue, and any combination thereof. In some aspects, the base comprises at least two base modules, wherein the at least two base modules are configured for insertion through an arthroscopic cannula and are further configured for assembly into the base after insertion. In some aspects, the at least two base modules are individually affixed to the second tissue using at least one suture, at least one anchor screw, or any combination thereof.

Other aspects of the disclosure are provided in additional detail below.

### DESCRIPTION OF THE DRAWINGS

Those of skill in the art will understand that the drawings, described below, are for illustrative purposes only. The drawings are not intended to limit the scope of the present teachings in any way.

FIG. 1 is an image of a python skull demonstrating the design and orientation of python teeth. The recurved teeth of pythons are designed to keep struggling prey in the python's grasp as the prey succumbs to constriction. As the prey struggles, its flesh is pulled closer to the snake's jawline, thus decreasing the chances of the prey escaping.

FIG. 2 is a schematic diagram illustrating a three-dimensional model of an array of tines on a biomaterial base. The array of recurved tines may serve to distribute stresses along a large number of attachment points, increasing the strength and toughness of the attachment.

FIG. 3 is a schematic diagram illustrating an in vivo rotator cuff repair using the python patch in one aspect. Pulling the supraspinatus tendon along the array of tines results in the tine array purchase of the tendon tissue, drawing it towards the base, which is secured to the humeral head via screws, sutures, or other standard methods.

FIG. 4 is a schematic diagram illustrating an in vivo rotator cuff repair using the python patch in another aspect. Tines are arrayed in opposite orientations on the biomaterial base. The tendon side array is composed of tines that grasp the tendon as the muscle generates tension while the tines on the opposite side grip the bone. In this configuration, the stiffness of the tines may be calibrated to match the stiffness of the tissue being grasped (i.e., the tines on the bone side may be stiffer than the tines on the tendon side).

FIG. 5A is an image of a 3-D printed array of tines on a base.

FIG. 5B is an image of a 3-D printed array of tines on a base.

FIG. 5C is an image of a 3-D printed array of tines on a base.

FIG. 5D is an image of a 3-D printed array of tines on a base.

FIG. 5E is an image of a 3-D printed array of tines on a base.

FIG. 6 is a diagram illustrating the convention for quantifying curvature in a tooth within an array.

FIG. 7 is an image showing a 2D stress model of a tooth in a tendon in accordance with one aspect of the disclosure. The orange line shows where the average stress was measured in the vertical direction (stress22). The white line shows the area that may be measured in this model. The area is shown in the results as the area over the total area possible along the length of the flat bottom part of the tendon.

FIG. 8 is an illustration of a spline model and a circle model used to define a 2D profile of a 2D stress model of the tooth.

FIG. 9 is an illustration of a conical 3D profile of a tooth used in a 3D stress model.

FIG. 10A is an image showing a 2D model of a 2D deformation (w′/w=1.5) of the 2D spline model of FIG. 8.

FIG. 10B is an image showing a 2D model of a 2D deformation (w′/w=1.5) of the 2D circle model of FIG. 8.

FIG. 11 is an image showing the max principal stress visualization displayed by Abaqus for the 2D spline model of the tooth for various values of w′/w. Abaqus averaged the stress from the elements if the stress was within 75%, i.e. if results from elements differ from others by more than 25% a discontinuity is displayed.

FIG. 12A is a graph summarizing the maximum principal stress estimated for the 2D spline model in Abaqus.

FIG. 12B is a graph summarizing the area of the tendon touching the tooth estimated for the 2D spline model in Abaqus.

FIG. 12C is a graph summarizing the stress in a vertical direction on the bottom face of the tendon estimated for the 2D spline model in Abaqus.

FIG. 13A is a graph summarizing the maximum principal stress estimated for the 2D circle model in Abaqus.

FIG. 13B is a graph summarizing the area of the tendon touching the tooth estimated for the 2D circle model in Abaqus.

FIG. 13C is a graph summarizing the stress in a vertical direction on the bottom face of the tendon estimated for the 2D circle model in Abaqus.

FIG. 14A is a wire-frame model illustrating deformation of a 3D tooth model with a 50° angle.

FIG. 14B is a 3D color map illustrating deformation of a 3D tooth model with a 50° angle.

FIG. 15A is a graph summarizing the maximum principal stress estimated for the 3D model in Abaqus.

FIG. 15B is a graph summarizing the area of the tendon touching the tooth estimated for the 3D model in Abaqus.

FIG. 15C is a graph summarizing the stress in a vertical direction on the bottom face of the tendon estimated for the 3D model in Abaqus.

FIG. 16 is a contour map showing an orthotropic stress distribution for the 2D circle model with w′/w=2.

FIG. 17A is a graph summarizing the maximum principal stress estimated for the 2D orthotropic model in Abaqus.

FIG. 17B is a graph summarizing the area of the tendon touching the tooth estimated for the 2D orthotropic model in Abaqus.

FIG. 17C is a graph summarizing the stress in a vertical direction on the bottom face of the tendon estimated for the 2D orthotropic model in Abaqus.

FIG. 18 is a schematic illustration of a standard double-row suture bridge repair of a rotator cuff.

FIG. 19 is a schematic illustration of a rotator cuff repair performed using a tooth array in accordance with one aspect of the disclosure.

FIG. 20 is a schematic diagram illustrating a variety of tooth shapes corresponding to a range of w′/w values.

FIG. 21 is a contour map of a representative FEM result of the interaction between a python tooth and a tendon showing stress (color map), contact area (white line), and average stress in the downward direction (orange lines).

FIG. 22 is a graph summarizing normalized contact area for tooth geometries of w′/w=1-3.5 obtained using the FEM of FIG. 21.

FIG. 23A is a graph showing representative force-displacement curves for a tooth with w′/w=0.5.

FIG. 23B is a graph showing representative force-displacement curves for a tooth with w′/w=2.5.

FIG. 24 is a graph summarizing the maximum force obtained after a displacement of 5 mm for teeth with w′/w ranging from 0.5 — 3.5 (* p<0.05, when compared to w′/w=0.5).

FIG. 25 is a graph summarizing a percentage of teeth engaging with a tendon during the force-displacement testing of FIG. 24, for teeth with w′/w ranging from 0.5-3.5.

FIG. 26 is a series of images illustrating a tooth shape and tooth arrays characterized by different tooth spacings.

FIG. 27 is a schematic illustration showing a tooth array device with patient-specific contouring.

FIG. 28A is a contour map illustrating the stress field within a tooth array device with a tooth spacing s=r

FIG. 28B is a contour map illustrating the stress field within a tooth array device with a tooth spacing s=2r

FIG. 28C is a contour map illustrating the stress field within a tooth array device with a tooth spacing s=3r

FIG. 29A is a graph summarizing forces of teeth at different horizontal positions within teeth arrays with different spacing patterns for a displacement corresponding to r/8. The colors of each trace correspond to different array spacings as denoted in FIG. 30.

FIG. 29B is a graph summarizing forces of teeth at different horizontal positions within teeth arrays with different spacing patterns for a displacement corresponding to r/2. The colors of each trace correspond to different array spacings as denoted in FIG. 30.

FIG. 30 is a graph summarizing total force as a function of elongation for tooth arrays with three different tooth spacings.

FIG. 31A is a graph of force versus elongation for tooth arrays with a tooth spacing equal to one tooth radius.

FIG. 31B is a graph of force versus elongation for tooth arrays with a tooth spacing equal to two tooth radii.

FIG. 31C is a graph of force versus elongation for tooth arrays with a tooth spacing equal to three tooth radii.

FIG. 32A is a graph of maximum force for tooth arrays with three different tooth spacings.

FIG. 32B is a graph of stiffness for tooth arrays with three different tooth spacings as assessed at 7 mm of displacement.

FIG. 32C is a graph of energy as assessed at 7 mm of displacement for tooth arrays with three different tooth spacings.

FIG. 33 is a schematic diagram illustrating a tensile test fixture used in shear lag tested as described herein.

FIG. 34 is a schematic diagram illustrating the tensile test fixture of FIG. 33 attached to a tendon.

FIG. 35 is a lattice porous base of the device, designed using nTopology element software in accordance with an aspect of the disclosure.

FIG. 36 is an illustration of a double-row repair performed using the device of FIG. 35.

FIG. 37 is a graph summarizing stress at tear-through for a range of single tooth geometries, normalized to the measured stress at w′/w=1.

FIG. 38 is a graph summarizing the contact area for a range of single tooth geometries, normalized to the measured contact area at w′/w=1.

FIG. 39 is a schematic diagram of a recurved tine in one aspect of the disclosure.

FIG. 40 is a top view of a tooth array device.

FIG. 41 is a bottom view of the tooth array device of FIG. 40.

FIG. 42 is an illustration of a double-row repair performed using the device of FIGS. 40 and 41.

FIG. 43A is a schematic drawing illustrating a first step of a surgical procedure using the device of FIGS. 40 and 41.

FIG. 43B is a schematic drawing illustrating a second step of a surgical procedure using the device of FIGS. 40 and 41.

FIG. 43C is a schematic drawing illustrating a third step of a surgical procedure using the device of FIGS. 40 and 41.

FIG. 43D is a schematic drawing illustrating a fourth step of a surgical procedure using the device of FIGS. 40 and 41.

FIG. 44A is a schematic drawing showing a first step of loading a suture through a suture hole using a suture puller.

FIG. 44B is a schematic drawing showing a second step of loading a suture through a suture hole using a suture puller.

There are shown in the drawings arrangements that are presently discussed, it being understood, however, that the present embodiments are not limited to the precise arrangements and are instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

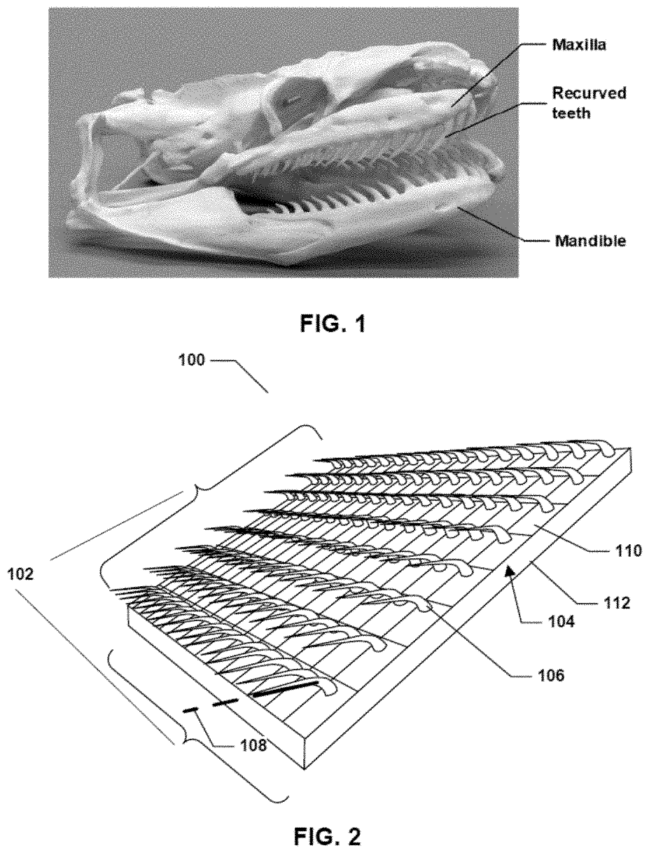

In various aspects, an orthopedic fixation device for attaching a first tissue to a second tissue is disclosed. In some aspects, the device is used as part of an orthopedic repair or reconstruction including, but not limited to, a repair or reattachment of a first tissue, such as a torn or ruptured ligament or tendon to a second tissue, such as a bone, ligament, or tendon. Without being limited to any particular theory, the disclosed device is inspired by the recurved teeth of pythons, illustrated in FIG. 1, that are designed to keep prey in the python's grasp. The prey is pulled along the long axis of the python's teeth and closer to the snake's mandibles and maxilla, thus decreasing the chances of escape. This bio-inspired method of grasping soft tissues using a plurality of recurved teeth arranged along the supporting surfaces of the mandibles and maxilla serves as a basis for the design of the orthopedic fixation device in various aspects, as described in detail below.

A schematic illustration of a fixation device 100 in one aspect is illustrated schematically in FIG. 2. The device 100 includes an array of tines 102 secured to a base 104. The base 104 interfaces directly with a hard or soft tissue surface including, but not limited to, tendon or bone. The array of tines 102 achieves fixation by means of a recurved design of each tine 106 of the array 102, which favors unidirectional traction of the tissue (not illustrated) along a tine axis 108 towards the base 104, thereby achieving a stable and robust grip.

In some aspects, the device 100 may include the array of tines 102 positioned on a first surface 110 of the base 104, as illustrated in FIG. 2. In these aspects, a first tissue 202 contacted with the array of tines 102 is affixed to the first surface 110 due to the penetration of individual tines 106 into the first tissue 202. In various aspects, the fixation of the first tissue 202 to the first surface 110 of the device 100 is enhanced by the advancement of the first tissue 202 along the tine axis 108 in response to tension forces from the first tissue 202 in a tissue pull direction 208. In these aspects, the base 104 is attached to a second tissue 210 using any suitable fastener including, but not limited to sutures, adhesives, and orthopedic fasteners such as screws, pins, or nails.

A fixation device 100a in a second aspect is illustrated schematically in FIG. 4. The device 100a includes a first array of tines 102 secured to the first surface 110 of the base 104 and a second array of tines 102a secured to the second surface 112 of the base 104. The tine axis 108a of the second array 102a is oriented opposite to the corresponding tine axis 108 of the first tine array 102. The first and second tine arrays 102/102a interface directly with hard or soft tissue surfaces including, but not limited to, tendon or bone. Each array of tines 102/102a achieves fixation by means of the recurved design of each tine 106 of the arrays 102/102a. In this aspect, the base 104 is secured to the first tissue 202 by the advancement of the first array of tines 102 into the first tissue 202 along the first tine axis 108. In addition, the base 104 is secured to the second tissue 210 by the advancement of the second array of tines 102a into the second tissue 210 along the second tine axis 108a. In some aspects, the attachment of the base 104 to the second tissue 210 is supplemented using any suitable fastener including, but not limited to sutures, adhesive, and orthopedic fasteners such as screws, pins, or nails. In various aspects, any of the devices described herein may further include a coating comprising an adhesive.

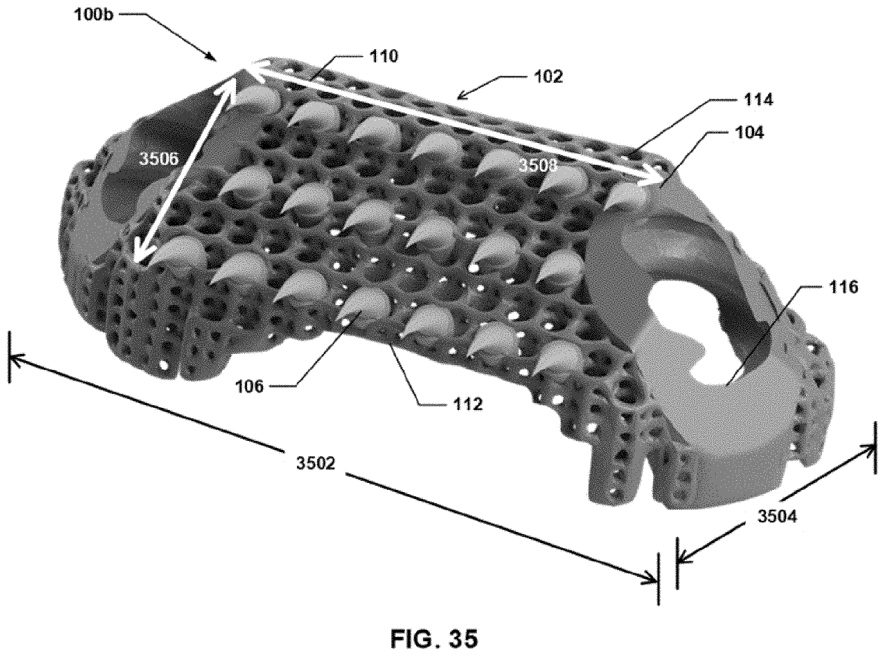

A fixation device 100b in a third aspect is illustrated schematically in FIG. 35. The device 100b includes a first array of tines 102 secured to the first surface 110 of the base 104. The first surface 110 is contoured to enhance the fixation achieved by the recurved tines 106 of the array 102 as described above. In this aspect, the second surface 112 is contoured to conform to the second tissue surface, thereby enhancing the contact and associated transfer of forces between the device 110b and the second tissue. Optionally, a second array of tines (not illustrated) oriented in a direction opposite to the first array of tines 102 may be provided on the second surface 112 of the device 100b to provide a means of securing the device 110b to the second tissue as described above.

Referring again to FIG. 35, a plurality of pores 114 are defined within the material of the base 104. The sizes and distribution patterns of the plurality of pores 114 mediate at least one or more functional properties of the device 100b including, but not limited to, the distribution and transfer of loads between the first tissue, device 100b, and the second tissue, as well as biointegration processes such as neovascularization and integration of bone tissue.

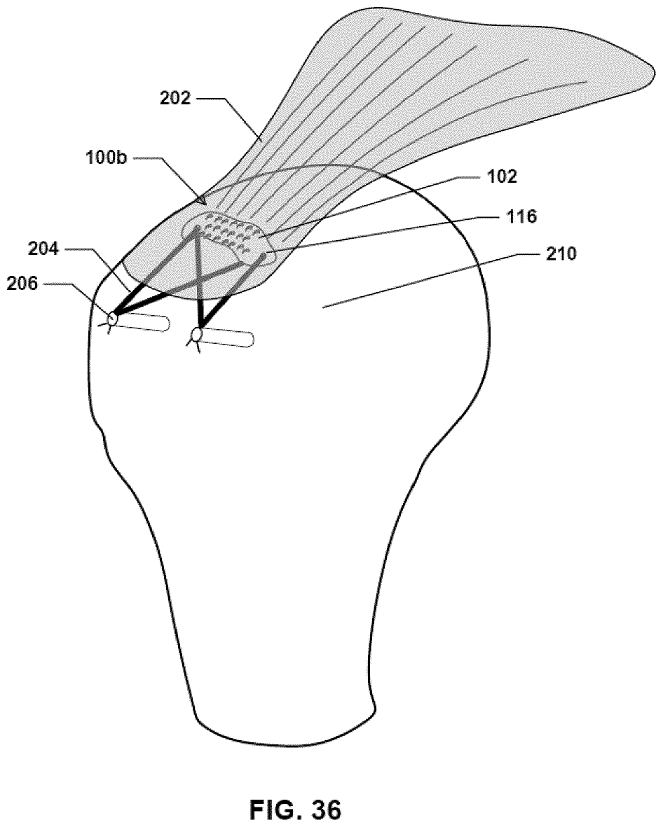

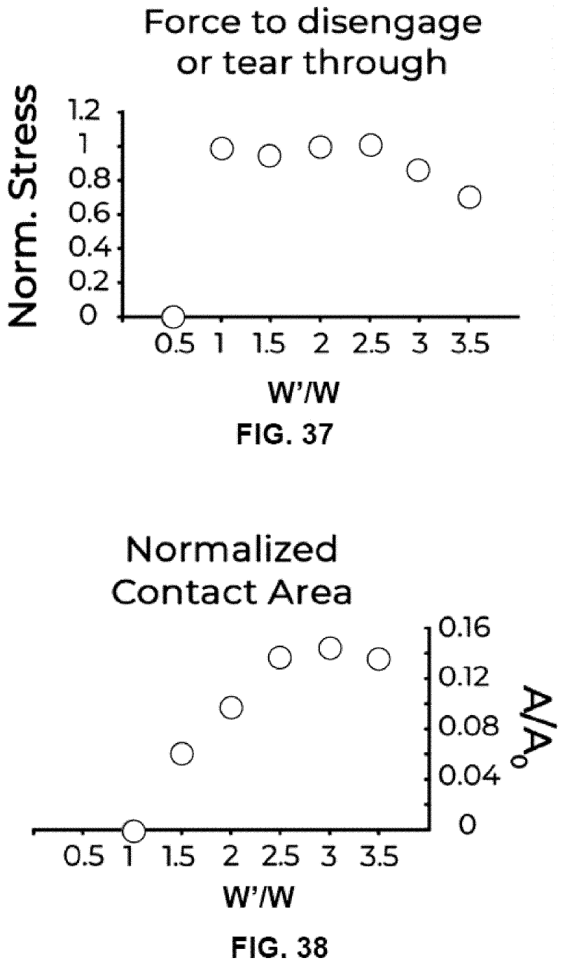

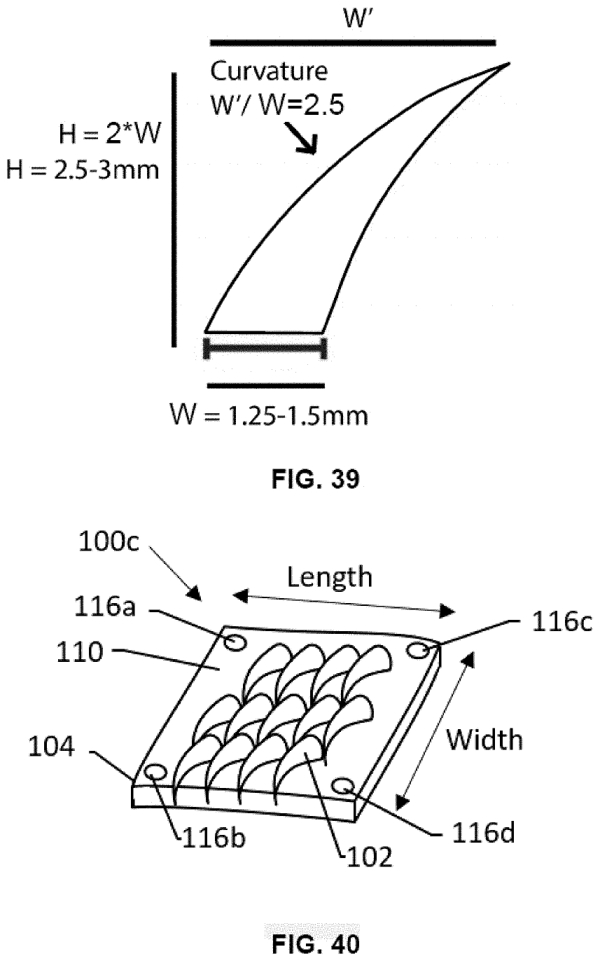

In addition, one or more fastener fittings 116 are formed within the material of the base 104, as illustrated in FIG. 35. Each of the one or more fastener fittings 116 are configured to receive any suitable fastener (not illustrated) used to attach the device 110b to the second tissue including, but not limited to, sutures, screws, nails, or staples. By way of non-limiting example, FIG. 36 schematically illustrates the device 100b attached to a second tissue 210 by sutures 204 passing through the fastener fittings 116; the sutures 204 are additionally secured to anchors 206 secured to the second tissue 210. In this non-limiting example, the first tissue 202 is secured to the array of tines 102 of the device 100b.

In various aspects, each fastener fitting is configured to receive a fastener including, but not limited to, any of the fasteners described herein. Each fastener fitting is further configured to secure, in cooperation with the fastener, at least a portion of the device to the second tissue.

In some aspects, the fastener fitting may be a hole or channel formed through a portion of the base and opening to the first and second surfaces of the base. In various aspects, the hole or channel may be configured to receive any suitable fastener without limitation. Non-limiting examples of suitable fasteners include sutures, staples, adhesives, and orthopedic fasteners such as anchor posts, screws, pins, or nails. In some aspects, the hole or channel may have any suitable cross-sectional profile without limitation. Non-limiting examples of suitable cross-sectional profiles include circular, oval, elliptical, polygonal, slotted, and any other suitable cross-sectional profile. In some aspects, the polygonal shape can be triangular, square, rectangular, and any other higher-order polygonal profile without limitation. In some aspects, the cross-sectional profile of each slot may be symmetrical or non-symmetrical.

In various aspects, the fastener fitting may be threaded or non-threaded. The minimum size of the fastener fitting is configured to minimally accommodate a fastener. In the case of a suture, the minimum size of the fastener fitting is configured to accommodate the diameter of the suture with sufficient clearance to provide for suture tightening by pulling the suture through the fastener fitting. In some aspects, the fastener fitting can range in diameter from about 0.15 mm to about 15 mm. In some aspects, the fastener fitting can range in diameter from about 0.75 mm to about 3 mm. In an exemplary example, the maximum diameter of the fastener fitting is about 6 mm. In some aspects, the cross-sectional profile of the fastener fitting may be matched to a corresponding cross-sectional profile of the fastener. Non-limiting examples include matched circular profiles to reduce shifting of the base once it is secured, as well as matched non-circular profiles to reduce shifting and rotation of the base. In some aspects, the non-circular profile may be a triangle, square, or any other polygonal profile without limitation.

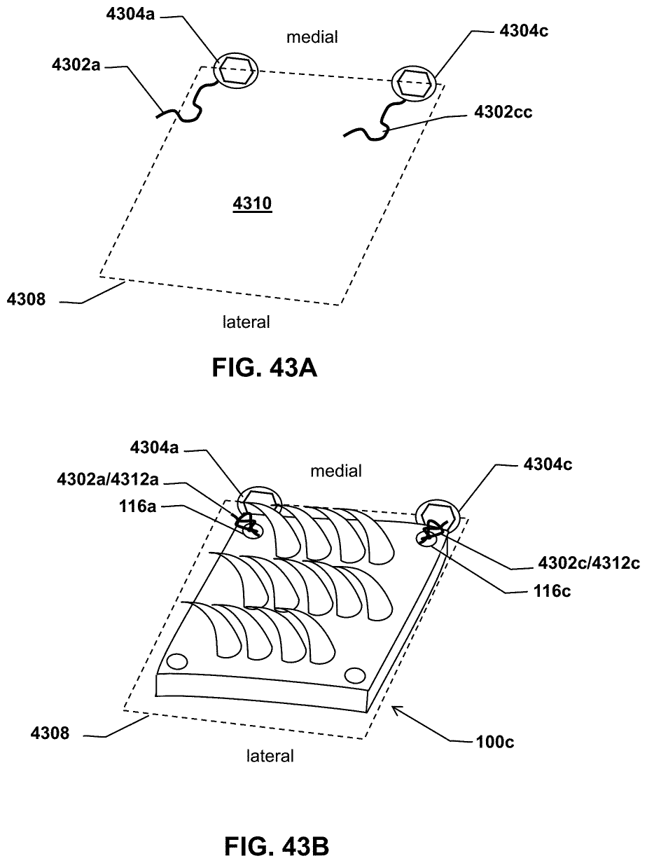

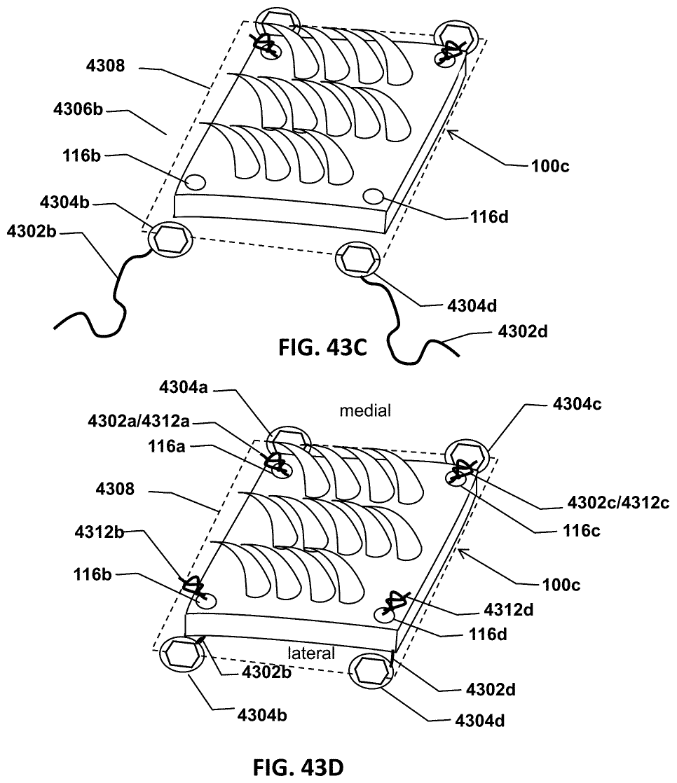

In some aspects, the device may be secured to the second tissue using sutures as fasteners. For securing the device to hard second tissues such as bone, the sutures are typically fastened to the bone by suture anchors mechanically fastened to the bone. In various aspects, the sutures used to secure the device to the second tissue may be fastened at one end to at least one suture anchor fastened to the bone. In some aspects, each suture may be fastened to a separate suture anchor, as illustrated in FIG. 43D. In other aspects, two or more suture anchors may be fastened to a shared suture anchor, as illustrated in FIGS. 43A and 43B (FIGS need to be revised).

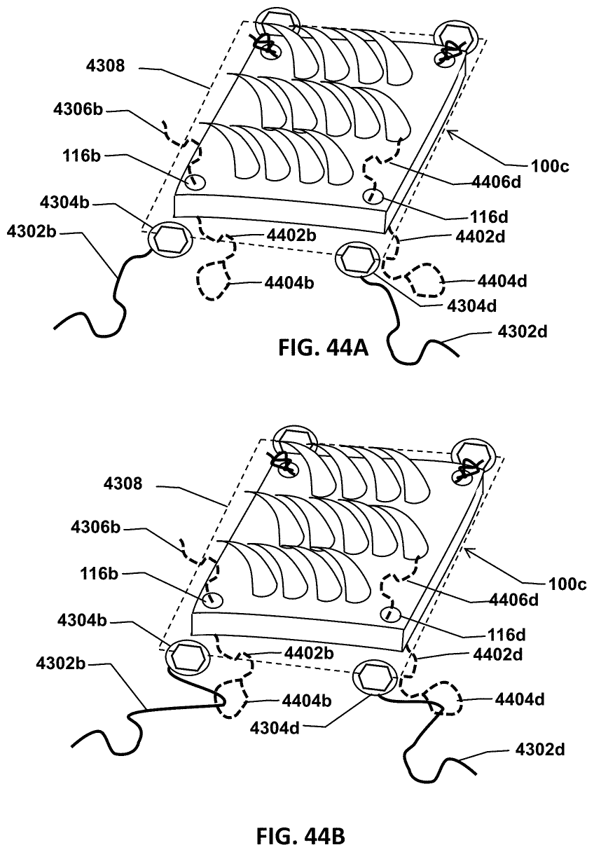

In various aspects, the sutures used to secure the device to the second tissue may be attached to the device using any suitable means without limitation. In one aspect, the free end of the suture can be inserted through a hole of the device and knotted with a slip knot. In other aspects, the suture may be looped through a hole in the device and the free end secured to a suture anchor. In other additional aspects, at least a portion of the sutures may be threaded through the fastener fittings using a suture hook thread or suture passer including, but not limited to, a Hewson suture retriever. FIGS. 44A and 44B illustrate a suture passer and the process of passing a suture through a fastener fitting in one aspect.

As illustrated in FIG. 44A, suture passers 4402b and 4402d may be preloaded through suture holes 116b and 116d prior to positioning device 100c within the desired device footprint 4308 with looped ends 4404b and 4404d protruding toward the suture anchors 4304b and 4304d and free ends 4406b and 4406d protruding from the opposite end of suture openings 116b and 116d. Referring to FIG. 44B, the sutures may then be loaded through suture holes 116b and 116d by passing sutures 4302b and 4302d through looped ends 4404b and 4404d of the suture passers 4402b and 4402d. The free ends 4306b and 4306d of the suture passers 4402b and 4402d may then be pulled to guide sutures 4302b and 4302d through the suture holes 116b and 116d. In some aspects, the free ends 4406b and 4406d of the suture passers 4402b and 4402d may be provided with handles, knots, grips, or other features to facilitate gripping and pulling the suture passers and loaded sutures through the suture holes.

In some aspects, sutures are anchored to a hard material with suture anchors. In some embodiments, the hard material is bone. In some aspects, sutures are looped through the soft material and pulled to place the soft material in contact with the fixation device. In some embodiments, the soft material is a tendon. Anchors may be any suitable existing anchor including any suture anchor known in the art. Anchors may be provided separately or may be provided along with existing systems or devices used for fixation. Non-limiting examples of suitable fixation systems include the Speedbridge™ implant system (Arthrex, Inc.), and non-limiting examples of suitable suture anchors include SwivelLock® suture anchors (Arthrex, Inc.). One non-limiting example of an existing anchoring system and device is described in Arthrex SpeedBridge™ Repair. vols AN1-0219-EN|Version: C, the content of which is incorporated by reference in its entirety.

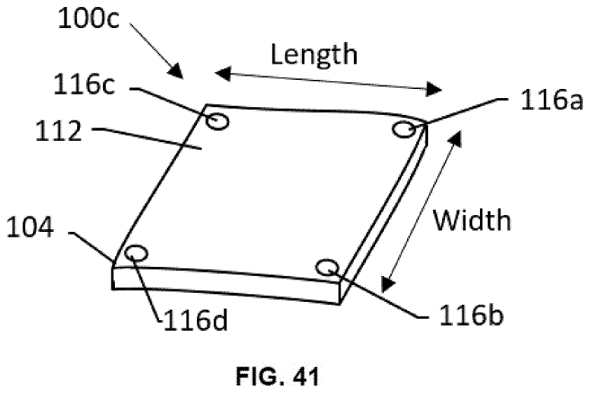

A fixation device 100c in a fourth aspect is illustrated schematically in FIGS. 40 and 41. The device 100c includes a first array of tines 102 secured to the first surface 110 of the base 104. Optionally, a second array of tines (not illustrated) oriented in a direction opposite to the first array of tines 102 may be provided on the second surface 112 of the device 100c to provide a means of securing the device 110c to the second tissue as described above.

In some aspects, a plurality of pores (not illustrated) may be defined within the material of the base 104. The sizes and distribution patterns of the plurality of pores mediate at least one or more functional properties of the device 100c including, but not limited to, the distribution and transfer of loads between the first tissue, device 100c, and the second tissue, as well as biointegration processes such as neovascularization and integration of bone tissue.

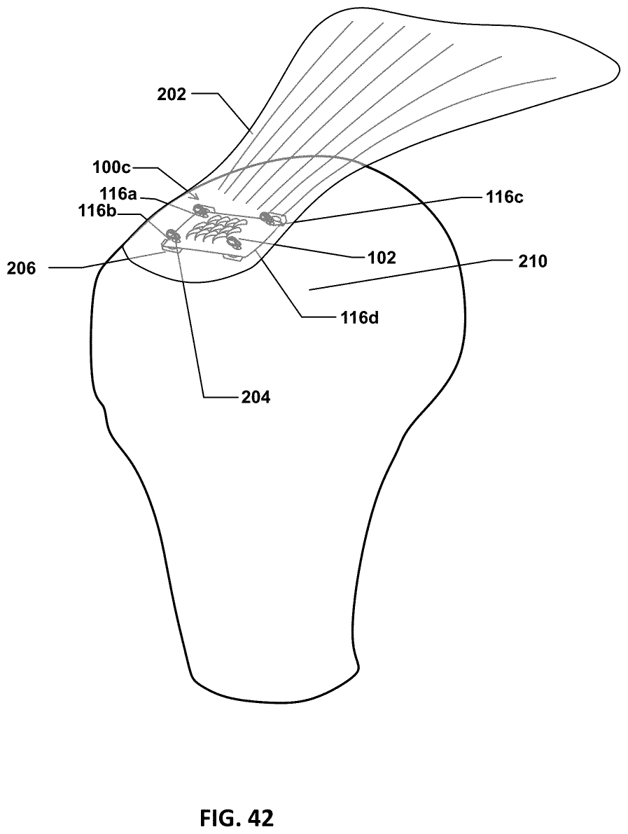

In addition, four fastener fittings 116a, 116b, 116c, and 116d are formed within the material of the base 104. Each of the fastener fittings 116a, 116b, 116c, and 116d are configured to receive one or more sutures used to attach the device 110c to the second tissue. By way of non-limiting example, FIG. 42 schematically illustrates the device 100c attached to a second tissue 210 by sutures 204 passing through the fastener fittings 116a, 116b, 116c, and 116d; the sutures 204 are additionally secured to anchors 206 that are secured to the second tissue 210. In this non-limiting example, the first tissue 202 is secured to the array of tines 102 of the device 100c.

By way of non-limiting example, a rotator cuff-specific device similar to the device 100c illustrated in FIGS. 40 and 41 consists of an optimized array of teeth placed in a curved, flexible base that matches the curvature and dimensions of the humeral head supraspinatus tendon attachment site. The base is of a rectangular shape and can be made in various sizes to accommodate for variations in the size of the attachment footprint across patients. Referring again to FIG. 40, the width of the base (medial/lateral) ranges from about 3 mm to about 9 mm, where each iteration may change in 0.5 mm increments (i.e., each variation of the device's width can be 3 mm, 3.5 mm, 4 mm, 4.5 mm, 5 mm, 5.5 mm, 6 mm, 6.5 mm, 7 mm, 7.5 mm, 8 mm, 8.5 mm and 9 mm). The length of the base (anterior/posterior) ranges from about 5 mm to about 18 mm, where each iteration changes in 0.5 mm increments (i.e., each variation of the device's length can be 5 mm, 5.5 mm, 6 mm, 6.5 mm, 7 mm, 7.5 mm, 8 mm, 8.5 mm, 9 mm, 9.5 mm, 10 mm, 10.5 mm, 11 mm, 11.5 mm, 12 mm, 12.5 mm, 13 mm, 13.5 mm, 14 mm, 14.5 mm, 15 mm, 15.5 mm, 16 mm, 16.5 mm, 17 mm, 17.5 mm, and 18 mm). Although the most commonly used device is likely to have a base width of 8 mm and a base length of 17.5 mm, other combinations of width and length as described above are available for selection by the surgeon for patient-specific use.

In some aspects, the base of the device is porous. The lattice structure of the base may be created using any suitable structural design software including, but not limited to, the software nTopology Element. The lattice shape may be any suitable shape without limitation. In some aspects, the lattice shape is hexagonal and is only applied at the base, and not on the teeth or close to the threads for the sutures.

In various aspects, the disclosed fixation device is used to splice or join first and second tissues, to affix a first tissue to a second tissue, and any combination thereof. The first tissue and second tissue are selected from any suitable soft or hard tissue without limitation. Non-limiting examples of suitable tissues that may be spliced, joined, and/or affixed include muscle tissue and connective tissues such as bone tissue, ligaments, tendons, cartilage, meniscus, fibrocartilage tissue, and adipose tissue. In some aspects, a soft tissue including, but not limited to, ligament, tendon, cartilage, meniscus, fibrocartilage, and adipose tissue is affixed to a hard tissue including, but not limited to, bone tissue. In other aspects, two soft tissues are spliced or joined together using the disclosed device.

In various additional aspects, the disclosed device is used to repair, reattach, and/or rebuild an attachment of a tendon or ligament to an insertion on a bone surface. In some aspects, the disclosed device is used to repair or rebuild at least a portion of a joint including, but not limited to an ankle joint, a knee joint, or a shoulder joint. Non-limiting examples of suitable repairs of ankle joints using the disclosed device include Achilles tendon repairs. Non-limiting examples of suitable repairs of knee joints using the disclosed device include reattachment of knee ligaments to a tibia and/or fibula. Non-limiting examples of suitable repairs of shoulder joints using the disclosed device include rotator cuff repairs such as reattachment of shoulder ligaments to a humeral head.

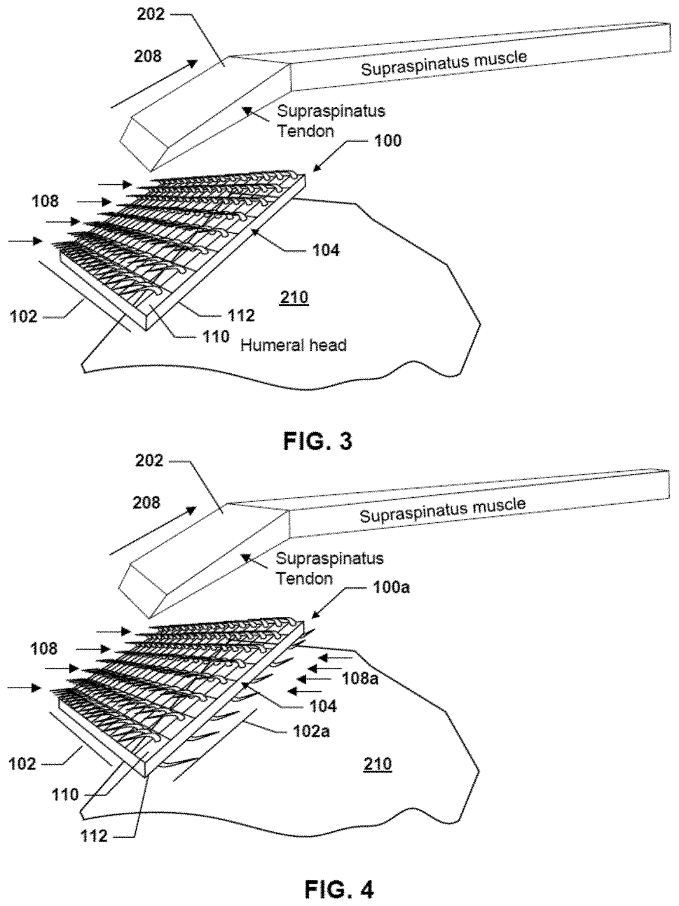

By way of non-limiting example, the disclosed device 100 is used to reconnect a supraspinatus tendon to the surface of the humeral head, as illustrated in FIG. 3. In this non-limiting example, at least a portion of the supraspinatus tendon is contacted with the array of tines 102 on the first surface 110 of the base 104, and the base 104 is attached to the humeral head by at least one orthopedic fastener such as an orthopedic screw, as illustrated in FIG. 19. In this non-limiting example, tensile forces 208 created by contraction of the supraspinatus muscle pull the supraspinatus tendon along the axis 108 of the tine array 102, reinforcing the robust attachment of the supraspinatus tendon to the tine array 102.

Referring again to FIG. 4, in another non-limiting example, the device 100a that includes first and second arrays of tines 102/102a positioned on both sides 110/112 of the base 104 is used to reconnect a supraspinatus tendon to the surface of the humeral head. In this non-limiting example, when the device 100a is positioned between the soft tissue and bone, the first and second arrays of tines 102/102a, which are oriented in opposite directions, form a strong tensile attachment. Tension forces 208 generated by contraction of the supraspinatus muscle advance the first tine array 102 into the supraspinatus tendon along the first tine axis 108, and the tension forces further advance the second tine array 102b into the humeral head along the oppositely-directed second tine axis 108b.

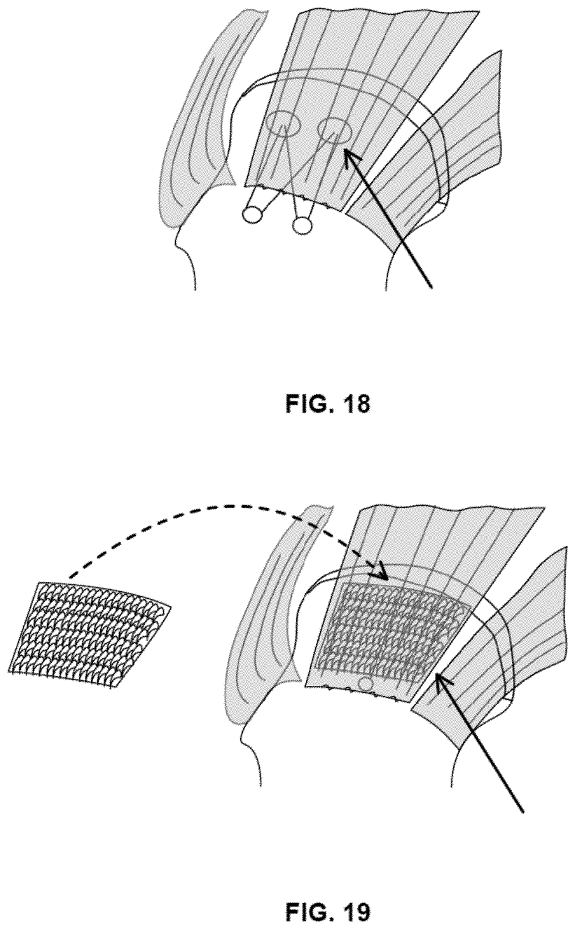

Without being limited to any particular theory, the integration of the tines with the tissues and the base form a stable soft tissue-hard tissue interface that mitigates potentially damaging stress concentrations typically associated with current surgical techniques, such as suture grasping and suture anchors, as illustrated in FIG. 18.

In various aspects, the base 104 of the disclosed device 100 provides a supporting foundation for a first array of tines 102 on a first surface 110, as illustrated in FIG. 3 and optionally provides a supporting foundation for a second array of tines 102a on a second surface 112, as illustrated in FIG. 4. In various other aspects, the base 104 distributes the forces acting at first and second tissues 202/210 attached to the device 100 over at least a portion of the surfaces 110/112 of the device 100. In various additional aspects, the base is optionally provided with additional features, including, but not limited to lattices of pores as illustrated in FIG. 35, to enhance at least one biological function associated with healing including, but not limited to, neovascularization.

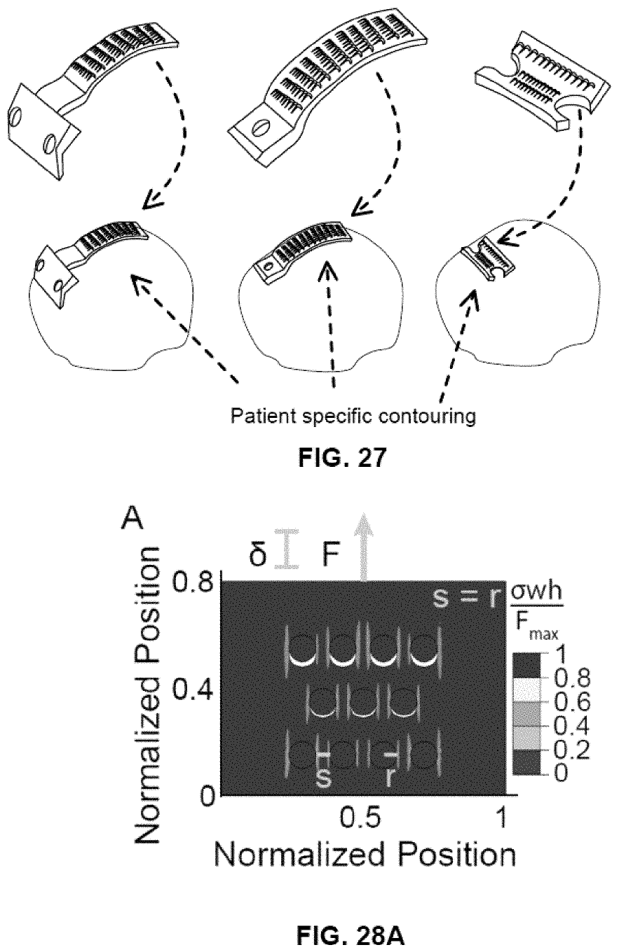

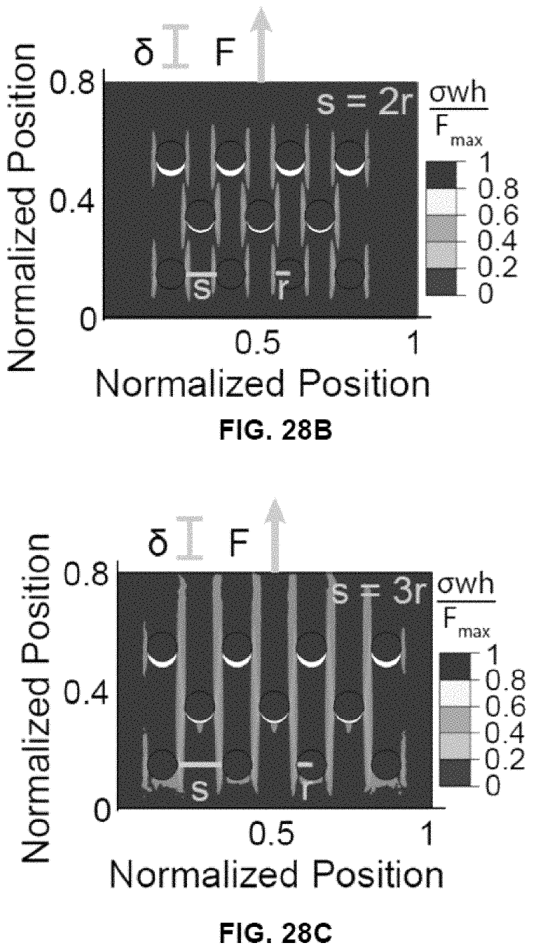

In some aspects, the base may be planar, as illustrated in FIGS. 3 and 4. In other aspects, the base is contoured to enhance the contact of the second surface with the second tissue to which the device is attached, as illustrated in FIG. 27 and FIG. 35. In other additional aspects, the base is contoured to conform to a contour of the second tissue surface of an individual patient, as illustrated in FIG. 27.

Without being limited to any particular theory, the base is designed to receive and distribute tensile forces from the attached first tissue and to transfer and distribute these tensile forces to the attached second tissue. In various aspects, the base is formed from a material with a stiffness ranging from relatively rigid to relatively flexible. Without being limited to any particular theory, a rigid base material provides a robust structural support for arrays of tines on one or both surfaces of the base. In addition, robust fittings for fasteners including, but not limited to, staples, screws, nails, anchors, or sutures can be formed within a relatively rigid base material. Conversely, a relatively flexible material provides the ability to locally deform the base to compensate for irregularities in the surface of the second tissue to which the base is attached. In addition, relatively flexible material further provides elastic deformation and recovery during loading and unloading of the device by forces applied by the first and/or second tissues to reduce jerk (defined herein as the rate of change of acceleration with respect to time) and associated high instantaneous forces experienced by the device and attached tissues during use.

In some aspects, the structural stiffness of the base is relatively uniform. In other aspects, locally stiff portions of the base are separated by locally flexible regions to provide for limited deformation of the base to enhance the fit of the base to the contour of the second tissue surface. In other additional aspects, at least a portion of the base is provided in the form of a mesh. Without being limited to any particular theory, the mesh-like base material may facilitate integration of the two tissues joined using the disclosed device. In some aspects, at least a portion of the base may be coated with an orthobiologic coating or loading within the pores of the base that release slowly into adjacent tissues to augment healing over time. Non-limiting examples of suitable orthobiologic compounds include tissue growth factors, biologic drugs, PRPs, MSCs, and any combination thereof. In various aspects, the material properties of the base locally vary between discrete stiff and flexible regions. In various other aspects, the material of the base continuously varies over one or more stiffness gradients.

In some aspects, a plurality of pores may be defined within the material of the base. Each pore of the plurality of pores may have any suitable size or shape without limitation. In some aspects, the plurality of pores may be of uniform size and distribution. In other aspects, at least portions of the pores may vary in size and/or shape within one or more regions of the device. In some aspects, at least portions of the plurality of pores may be arranged in a distribution pattern selected from uniform distribution, rows, columns, and lattice distributions. Non-limiting examples of suitable lattice distributions include cubic, tet-oct, oct, quad prism, stochastic, and hexagonal lattice distributions. In one example, at least a portion of the plurality of pores is distributed in a hexagonal lattice pattern as illustrated in FIG. 35. Without being limited to any particular theory, the sizes and distributions of the plurality of pores within the base may be selected to control the distribution and transfer of loads between the device and attached tissues. In additional apects, the selected sizes and distributions of the plurality of pores within the base may be selected to modulate biointegration processes such as neovascularization and integration of bone tissue within the device and attached tissues. In other additional aspects, the selected sizes and distributions of the plurality of pores within the base may be selected to reduce the maximum stress of the device's base in use and/or to reduce the likelihood of mechanical failure of the device in use.

In various aspects, the device is constructed using any suitable biocompatible materials without limitation. Non-limiting examples of suitable biocompatible materials for the disclosed device include polymers, collagen, mineralized collagen, metals, minerally coated metals, ceramic-coated metals, and any combination thereof. In some aspects, at least portions of the device or the entire device may be constructed using biodegradable materials to promote biologic integration between tissues such as healing tendons and bones joined together using the device. In some aspects, the biocompatible materials of the base can possess uniform material properties throughout the material cross-section or can be designed to possess a functional gradient in material properties, further augmenting interfacial toughness.

In some aspects, the disclosed device includes an array of tines positioned on one surface of the base as described above and illustrated in FIG. 3. The pattern of tines can be uniform (as shown in FIGS. 2, 3, and 4) or the pattern of tines can be patterned to accommodate the expected stress field of the particular attachments of tissues in various aspects. In other aspects, the disclosed device includes a first array of tines and a second array of tines positioned on opposite surfaces of the base of the device, as illustrated in FIG. 4.

Without being limited to any particular theory, the distribution and shapes of the tines within each tine array are patterned to provide more uniform or less uniform stress distribution over the attached tissue and to provide for control of the “shear lag” effect as loads are transferred from tendon to bone using the disclosed device. In various aspects, each tine of each array is oriented such that the tension force produced by the tissue to be affixed to the device is pulled along the tine and toward the surface of the base supporting the tines. In various aspects, the tines of the first and second arrays of tines may be aligned in parallel to each other, oppositely aligned, and any alignment therebetween without limitation. In some aspects, individual tines within an array may all align along a single tine axis. In other aspects, individual tines within an array may have different alignments. In these other aspects, the different alignments of tines within an array may be selected to modulate stress distribution (i.e. reduce or minimize) over the device and/or reduce or equalize the stresses within the tissues attached to the tiny array.

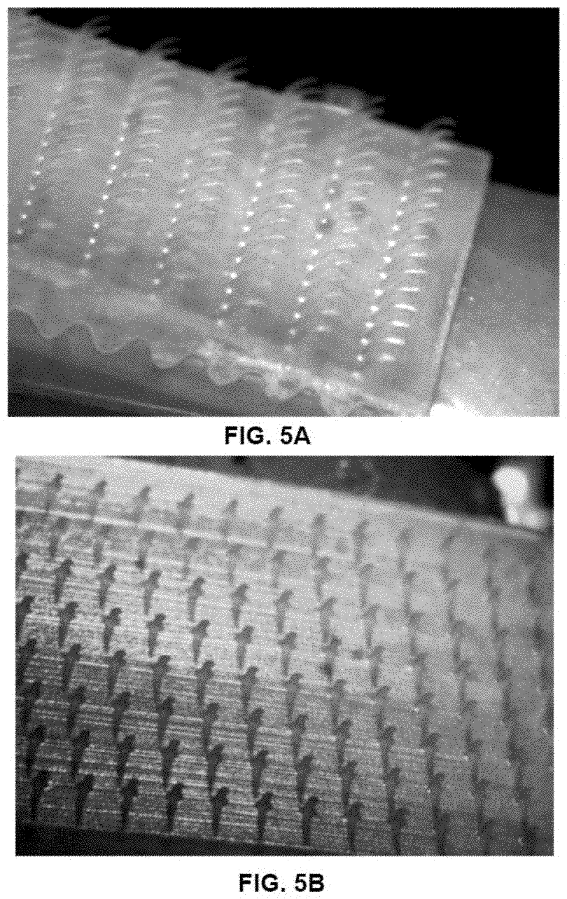

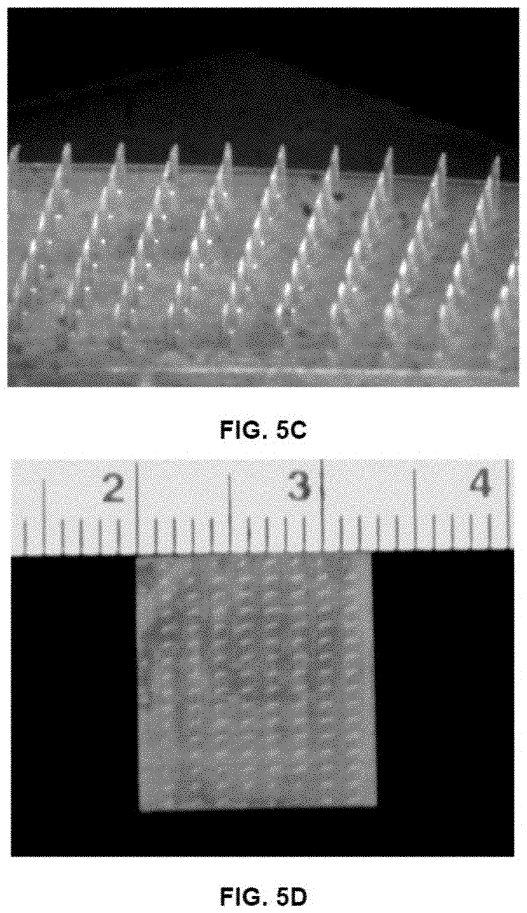

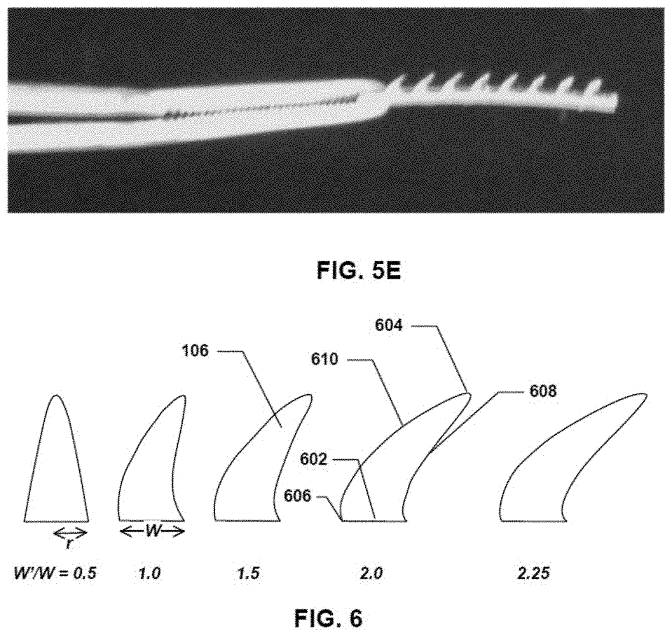

In various aspects, the tines within the array are arranged in an array pattern. The plurality of tines may be arranged in any suitable pattern without limitation. In some aspects, the tines are arranged in aligned rows and columns, as illustrated in FIG. 2. In other aspects, the tines are arranged in rows and offset columns, as illustrated in FIG. 26 and FIG. 35. In various aspects, the tine array includes any number of rows without limitation. In these various aspects, the tine array includes one row of tines, two rows of tines, three rows of tines, four rows of tines, five rows of tines, six rows of tines, seven rows of tines, eight rows of tines, nine rows of tines, ten rows of tines, or more. In various other aspects, each row of tines includes one tine, two tines, three tines, four tines, five tines, six tines, seven tines, eight tines, nine tines, ten tines, or more. Various non-limiting examples of tine arrays are illustrated in FIGS. 5A, 5B, 5C, 5D, and 5E.

In various aspects, adjacent rows within a tine array are separated by any suitable row separation distance without limitation. In some aspects, adjacent rows of tines within a tine array are separated by a distance d that is proportional to the maximum cross-sectional radius r of the tines, as illustrated in FIG. 26. In various aspects, the row separation distance d ranges from about 0.5r to about 5r. In various other aspects, the row separation distance d is 0.5r, 0.75r, r, 1.25r, 1.5r, 2r, 2.5r, 3r, 3.5r, 4r, 4.5r, or 5r.

In various aspects, adjacent tines within a row of tines in a tine array are separated by any suitable distance without limitation. Without being limited to any particular theory, the separation of adjacent tines in the tine array influence at least one or more parameters indicative of the performance of the tine array including, but not limited to, maximum tissue holding force, energy, stiffness, stress distributions, and any combination thereof. Non-limiting examples illustrating the sensitivity of maximum tissue holding force, energy, and stiffness to variations in tine spacing are provided in detail in the Examples below. In some aspects, adjacent tines within a row of tines in a tine array are separated by a tine separation distance s that is proportional to the maximum cross-sectional radius r of the tines. In various aspects, adjacent tines within a row of tines in a tine array are separated by a tine separation distance s that ranges from about 0.5r to about 5r. In various other aspects, the tine separation distance s is 0.5r, 0.75r, r, 1.25r, 1.5r, 2r, 2.5r, 3r, 3.5r, 4r, 4.5r, or 5r.

In various aspects, the shape and dimensions of the tines within the tine array influence at least one or more parameters related to tissue gripping performance including, but not limited to, stress within the tissue and contact area, force generation, and any other suitable tine performance parameter without limitation. As illustrated in FIG. 6, the tines 106 of the tine array are characterized by a cross-sectional profile and a curvature in various aspects. In various aspects, each tine includes a cross-sectional profile that includes a maximum cross-sectional diameter r at the base 602 of the tine positioned at the base surface that tapers to a pointed tip 604. In various other aspects, the curvature is characterized by the ratio w′/w, where w is the distance between the rear-most position 606 and forward-most position 608 of the tine 106 at the base 602, and w′ is the distance between the rear-most position 606 and tip position 604 of the tine at the base 602.

In various aspects, the ratio w′/w ranges from about 0.5 to about 5. In various other aspects, the ratio w′/w is 0.5, 0.75, 1, 1.25, 1.5, 2, 2.5, 3, 3.5, 4, 4.5, or 5. In one aspect, the ratio w′/w is 2.5. Without being limited to any particular theory, a tine with the ratio w′/w of 2.5 is thought to result in enhanced tissue stress and associated force production within an attached tissue, as illustrated in the examples below.

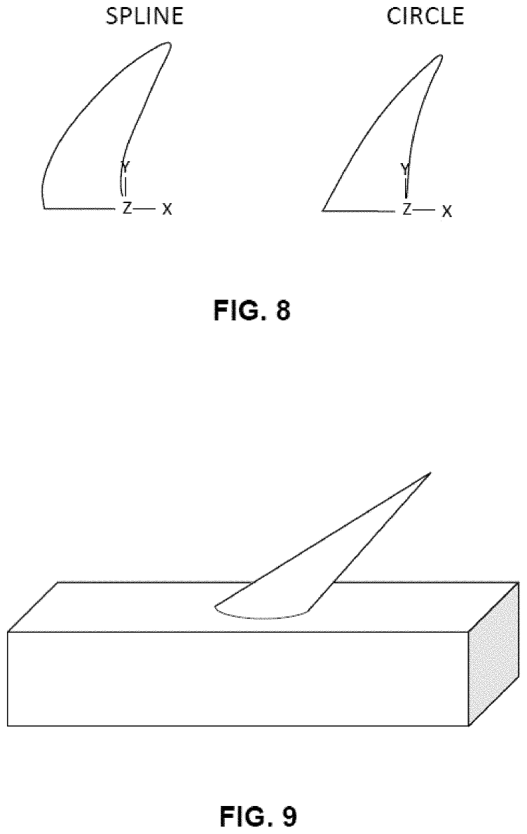

Referring again to FIG. 6, the exterior surface of the tine 106 may further be defined by a forward curve 608 and a rear curve 610. In some aspects, the forward and rear curves 608/610 are defined using a circular model, wherein the forward and rear curves 608/610 are defined as segments of circles with larger and smaller radii, respectively. In other aspects, the forward and rear curves 608/610 are defined using a spline model, wherein a spline fit formed through predefined points representative of the desired curvature is used to define the forward and rear curves 608/610. A comparison of forward and rear curves 608/608 defined using a circular model and using a spline model is provided in FIG. 8. Additional non-limiting examples of methods of defining the contours and curvature of the tines are provided in additional detail in the examples below.

In various aspects, the tines may be produced from any suitable biocompatible material without limitation. Non-limiting examples of suitable biocompatible materials for producing the tines include polymers, collagen, mineralized collagen, metals, minerally coated metals, and ceramic-coated metals. In some aspects, the tines may be constructed using biodegradable materials to promote biologic integration between the healing tissues joined using the device.

In some aspects, the devices are provided as a single base with arrays of tines positioned on one or more surfaces of the base as described above. In other aspects, the devices may be provided in the form of two or more base modules that may be independently positioned and secured to the second tissue using the fasteners as described herein. Without being limited to any particular theory, the two or more base modules may be sized to fit through an incision in the skin of a patient during surgery to facilitate the implementation of a surgical repair or fixation procedure using the device or devices described herein. In various aspects, the base modules may be positioned independently in any suitable location and orientation as needed to attach two tissues. Base modules may include a base with a single row of multiple tines, a single column of multiple tines, or any combination thereof. In various aspects, a single row or column may independently include at least one tine, at least two tines, at least 3 tines, at least 4 tines, at least 5 tines, at least 6 tines, at least 7 tines, at least 8 tines, at least 9 tines, and at least 10 tines or more. In various aspects, the spacing of individual tines from tines in adjacent rows or columns is similar to the tine spacing as the corresponding tine spacing of single-base devices as described hererin.

In some aspects, base modules may be positioned at different locations and orientations within the repair site and secured to the underlying second tissue independently. In some aspects, the use of multiple base modules provides for enhanced individualization of repair procedures. In some aspects, the base modules provide levels of tissue fixation that can vary spatially as needed to attach 2 tissue surfaces in a subject. In some aspects, the use of two or more base modules can reduce the size of incisions/repair sites due to the smaller size of elements.

In various aspects, the base modules may be configured to be assembled into a single device structure in situ after placement/insertion into the repair site as individual base units. In some aspects, the base modules may be assembled into a single device by independently securing each base module such that the modules are arranged as a single device. The base modules may be provided with additional attachment features that include mechanically interacting components configured to attach one module to an adjoining module in a predetermined pattern to form a single device. Non-limiting examples of suitable additional attachment features include matched tabs and slots, interdigitating protrusions and cutouts, and any other suitable mechanically interacting components without limitation.

In various aspects, the device is configured to be folded, rolled, or otherwise manipulated to fit through an arthroscopic cannula for implantation in an arthroscopic setting. In various other aspects, the device may be provided in the form of two or more base modules as described above. In some aspects, the base modules are sized and dimensioned to fit through an arthroscopic cannula for implantation in an arthroscopic setting. In other aspects, the base modules are constructed from a flexible material and are configured to be folded, rolled, or otherwise manipulated to fit through an arthroscopic cannula for implantation in an arthroscopic setting.

In various other aspects, the devices described above are produced using any suitable fabrication method known in the art without limitation. In one aspect, the device is produced using a 3D printing fabrication method. Without being limited to any particular method, 3D printing fabrication enables the production of devices that include bases contoured to conform to the contours of the tissue surfaces of individual patients, thereby enhancing the fit of the device to the tissues to be joined using the device as described above.

The devices described herein may be used to provide fixation or joining of tissues associated with any suitable musculoskeletal and other tissue interface anatomical sites without limitation. One non-limiting example of a suitable musculoskeletal site for which the device may be used to provide fixation or joining of tissues is the joining of a tendon to bone in a rotator cuff repair as described herein. In various aspects, the device may be used without further supplementation as described hererin to provide the fixation or joining of tissues. In various other aspects, the device may be used in combination with at least a portion of an existing fixation system and/or system elements, such as sutures and suture anchors, to provide the fixation or joining of the tissues. One non-limiting example of a suitable existing fixation system is described in Arthrex SpeedBridge™ Repair. vol. AN1-0219-EN|Version: C, the content of which is incorporated by reference in its entirety.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

### EXAMPLES

The following examples illustrate various aspects of the disclosure.

### Example 1

### 3D Printed Arrays of Tines on Biomaterial Bases

A virtual 3-D model of the array of tines on a biomaterial base was produced to demonstrate select possible orientations. Screen captures of this model are shown in FIG. 2, FIG. 3, and FIG. 4. This model was then manufactured using a 3-D printer. Photographic images of the 3-D printed arrays of tines produced in this experiment are shown illustrated in FIG. 5A, FIG. 5B, FIG. 5C, FIG. 5D, and FIG. 5E.

### Example 2

### Effects of Tooth Dimensions on Tooth Structural Properties

A virtual 3-D model of the reattachment of tendon to bone is a perennial surgical challenge because the sutures and anchoring systems used for reattachment can tear the soft tissue that they try to hold in place. Python teeth are effective at holding onto soft tissue without tearing it, perhaps optimized as a means of holding onto and ingesting their potentially squirming prey. The aim of this experiment was to explore the mechanics of python teeth as a model for holding tendons in place in surgeries. The curvature of python teeth versus the lack of curvature in something like a shark tooth, the latter presumably optimized for cutting rather than grasping, points to a correlation between the curve of a hook of a tooth and its ability to grasp. Different “tooth” geometries were explored in Abaqus to gain insight into how the shape of a tooth affects the stress and deformation in a highly simplified model of biting. Two different tooth geometries were explored parametrically in two dimensions (2D), and a third was explored in 3D. Isotropic and orthotropic hyperelastic material properties were considered. Results suggested that tooth curvature could be optimized to reduce tearing stresses and improve tooth/gum contact stresses when the “tendon” is stressed.

Injuries to the shoulder are widespread and often require surgical repair. Musculoskeletal injuries are a leading cause of pain and disability and result in significant health care costs. Approximately 30% of the population over the age of 60 has a rotator cuff tear at the tendon-to-bone insertion site. Tens of thousands of rotator cuff repairs are made every year. Healing after rotator cuff repair is a well-known clinical challenge. Failure rates range from 20% to 94%.

It is believed that part of this failure rate results from the stress caused by moving the arm is too focused due to the current approach used to repair the tendons. A healthy interface between the soft anisotropic tendon and the hard nearly isotropic bone is accounted for by a gradient region from tendon to bone showing gradual increases in mineral content and collagen misalignment. Normally there is a complex, millimeter-sized transitional tissue with gradations in structure to help account for the structural mismatch between the tendon and bone. In the absence of this scaffold, the stress created when moving the arm is mostly placed on the sutures used to keep the tendon in place after surgery. However, the suture acts like a knife and can cut through the tendon rather than keep it in place. A device to distribute the stress across the tendon like the original scaffold may increase the success rate of rotator cuff surgeries.

In the animal kingdom, examples of interactions of the same sort can be found in the teeth of carnivores. Animals have different tooth geometries based upon how they kill or eat their prey. Sharks have triangular-shaped teeth pointed mostly up with very little curve. Their teeth are meant for cutting because they take bites out of their prey. Unlike the shark, the tooth of a python is curved. They swallow their prey and need to latch on. The hooked teeth keep the prey from getting away and allow the python to grip soft tissue without it tearing. This principle can be used to keep a tendon in place after surgery.

Abaqus was used to model the tooth in the tendon. The parts were first modeled in two dimensions. The first set of parts was created by using the spline feature in Abaqus with four points being picked each time, (−1,0), (x,4), (1,0), and (4, −1). X was changed each time to adjust the curvature of the tooth. Note that length units are in general arbitrary in solid mechanics in the absence of surface energy effects such as cohesion.

The curvature was tracked by using w′/w as shown in FIG. 6. A frictionless contact interaction was created between the tooth and the tendon for simplification. There was a hard contact penetration penalty for the contact interaction. The contact interaction has no pressure on the surfaces until contact, and after contact, there is a nonlinear increase in pressure between the surfaces. The load was applied to the upper right node of the tendon, and the rest of the nodes on the right side were locked to move the same amount as the upper right node so the load was effectively distributed across the right face. The mesh was refined at the point of the tooth until the resulting visualization of the stress showed a gradual radiating stress. The degrees of freedom were taken away from the left and right edges of the tendon so that they moved in unison. This allowed for the model to act as if the tooth was in the middle of a field of teeth instead of just by itself.

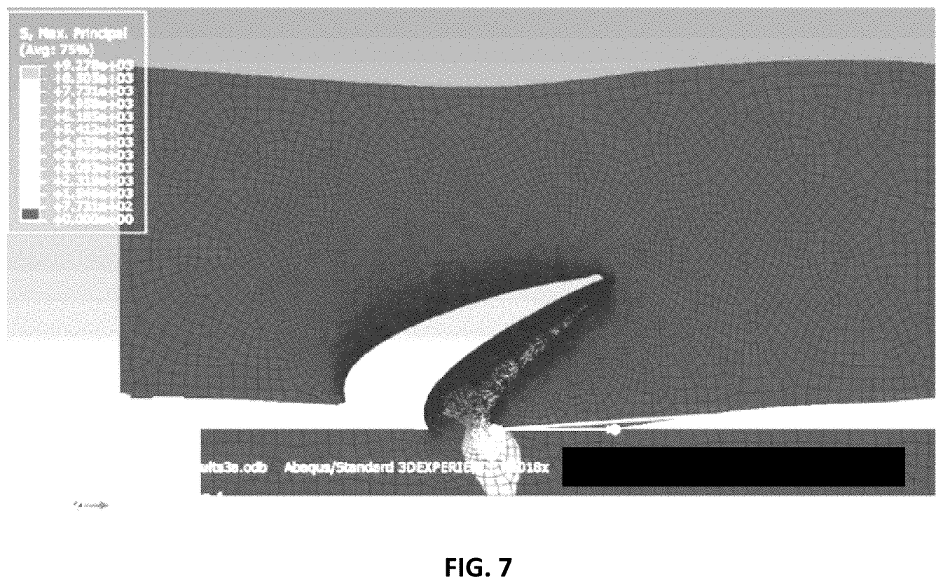

Several parameters were measured and recorded from the results of the model. The maximum principal stress was recorded from each model to show which shape is most likely to tear or break the tendon due to stress. The area of the flat part of the bottom of the tendon that was touching the flat part of the top of the tooth and the average of the stress in the downward direction (stress22) on the flat part of the bottom of the tendon was also measured and show how well the tooth can keep the tendon in place. FIG. 7 is included to help explain where the other parameters were measured.

The 2D model was also done with a shape that used two circles to make the tooth, as shown illustrated in FIG. 8A. A smaller circle with a radius of 9.25 for the inside of the tooth and a larger circle with a radius of 11.75 for the outside of the tooth. Solidworks software was used for the design. The circles were moved each iteration to adjust the w′/w value. The model was then extended to 3D with a cone shape, shown illustrated in FIG. 9. Orthotropic properties were added to the tendon in the 2D models with the circle shapes. Engineering constants were put into Abaqus to define the orthotropic properties which are shown in Table 1. Poisson's ratio was used in the 2D model based on published values, but the elastic modulus and shear modulus were selected to get the model to converge but still keep the maximum amount of orthotropic properties possible. The force was applied as a displacement on the right face of the tendon (or the face facing the point of the tooth) for the orthotropic models to help convergence. The 2D models were measured for w′/w=1-3.5. The cone model was changed each time by changing the angle of the cone. Five angles were used: 35°, 40°, 45°, 50°, and 55°.

TABLE 1
Orthotropic properties used in 2D circle model

| Parameter | E1 | E2 | Nul2 | G12 |
|---|---|---|---|---|
| Value | 1 | 0.2 | 0.0003 | 0.1 |

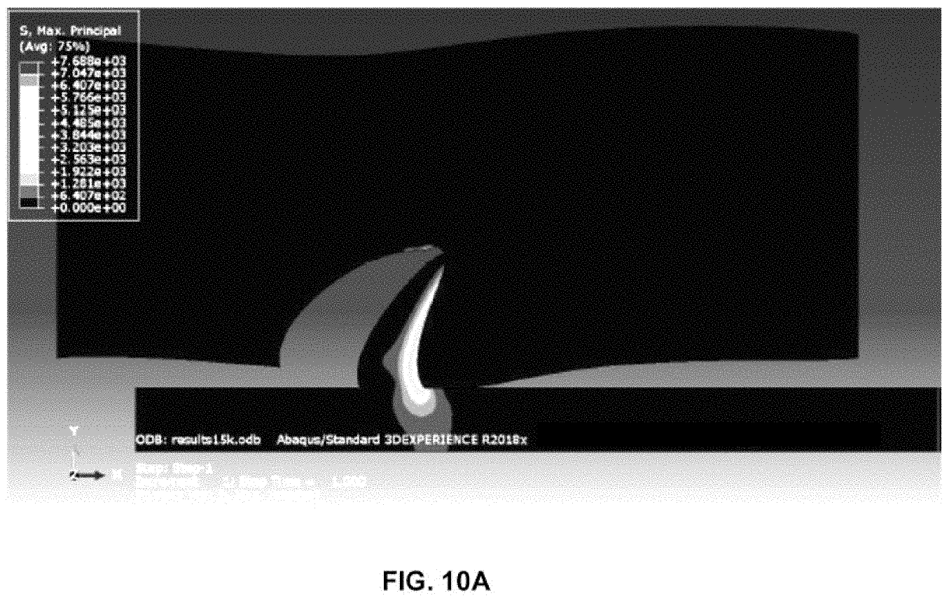

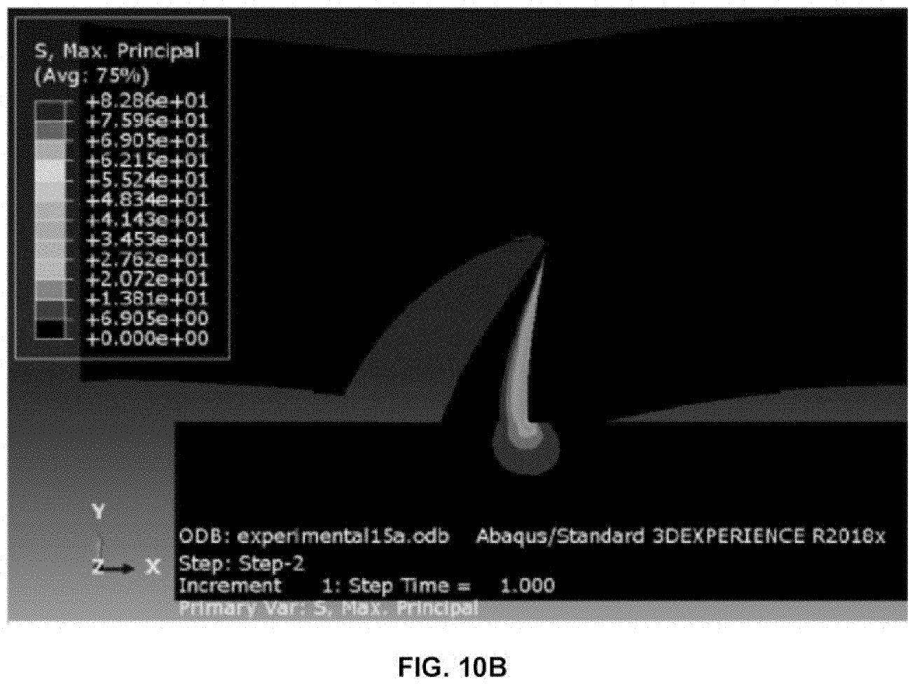

All the other parameters such as mesh and friction were kept constant across the changes in w′/w for consistency. Each model was processed in Abaqus and the results were recorded. The figures show some typical plots with deformation. We first asked how the tooth resisted stress and found that the stresses were highest at the base of the tooth for all shapes and were so high that the stresses in the tissue were typically all within the first contour level of stresses within the tooth (FIG. 10A and FIG. 10B). However, the material in the tooth should be strong enough to resist breaking. Therefore, the results were focused on stress in the tendons.

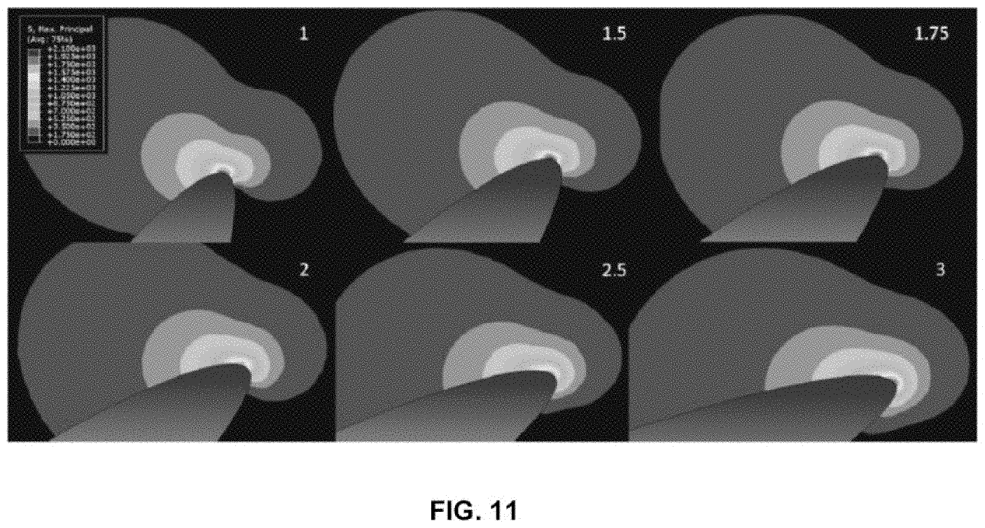

Abaqus showed a visualization of the stress. As a next step, the effect of tooth shape on the stress in the tendons was characterized. The tooth was removed in FIG. 11 to better observe the effects of the stress on the tendon.

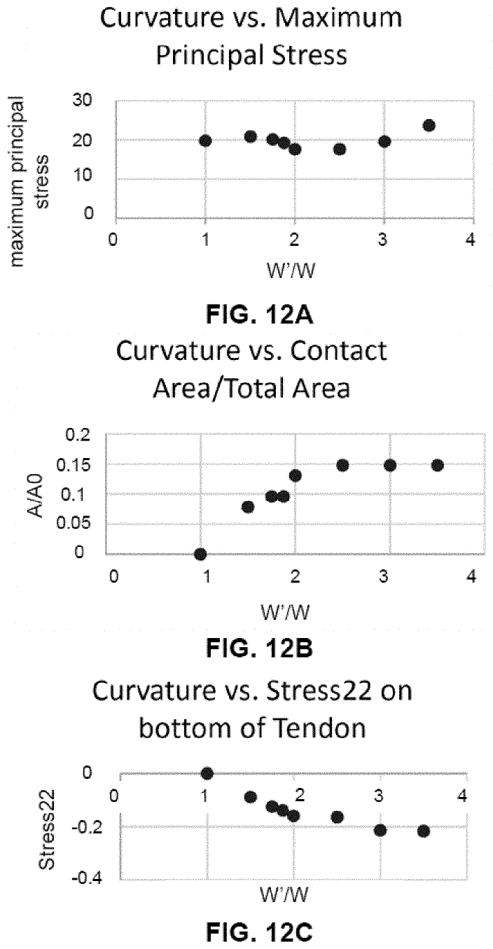

The models were run multiple times for different geometries and the maximum principal stress, the area of the tendon touching the bone over the total area on the bottom of the tendon, and the average of the stress on the bottom of the tendon were measured. This was done for both the spline model and the circle model. Convergence was achieved with models that had on the order of 25000 nodes. The results for the 2D spline model are shown in FIG. 12A, FIG. 12B, and FIG. 12C.

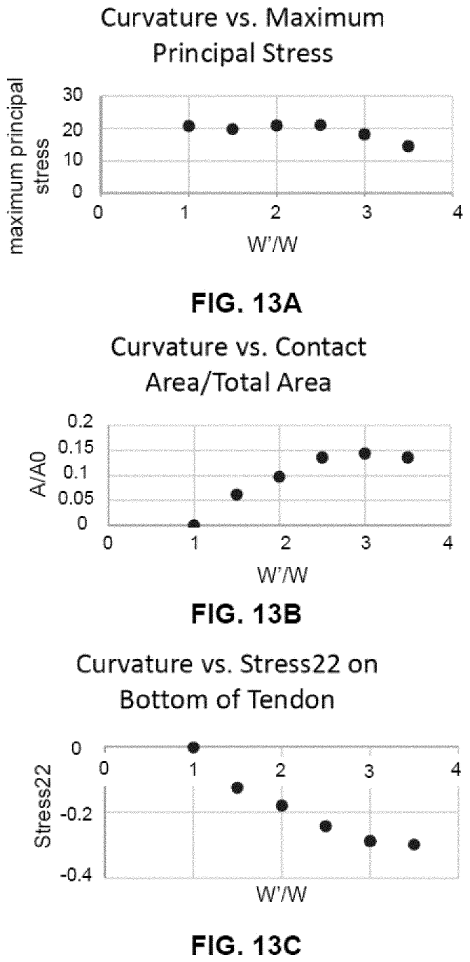

Next, the effect of different tooth shapes on the stress in the tendon was tested. In addition, models were created that may be easily tested through 3D printing the shapes for experiments. The results for the 2D circle model are shown in FIG. 13A, FIG. 13B, and FIG. 13C.

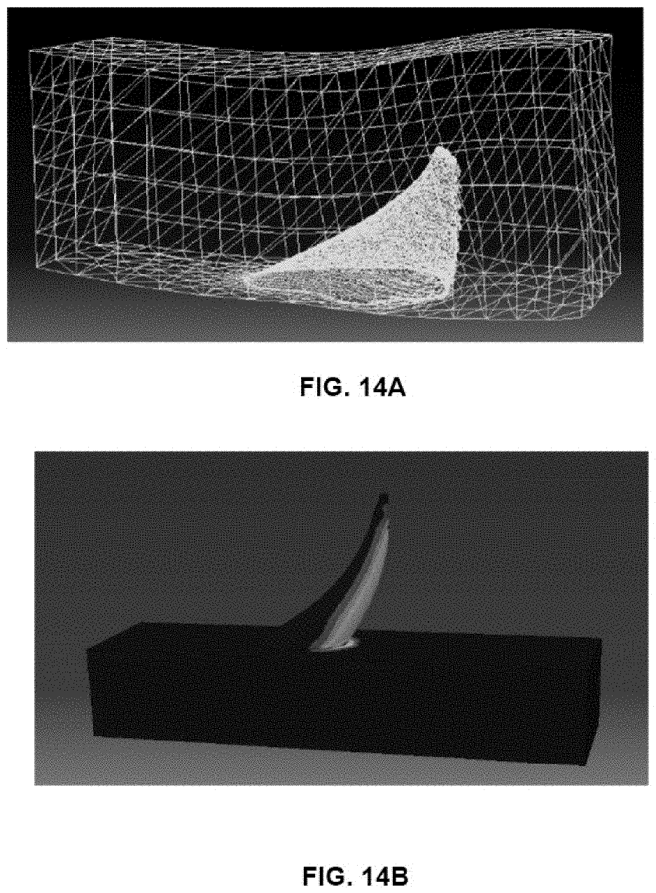

The model was then advanced into 3D. A cone shape was used for the geometry. Typical deformations are shown in FIG. 14A and FIG. 14B.

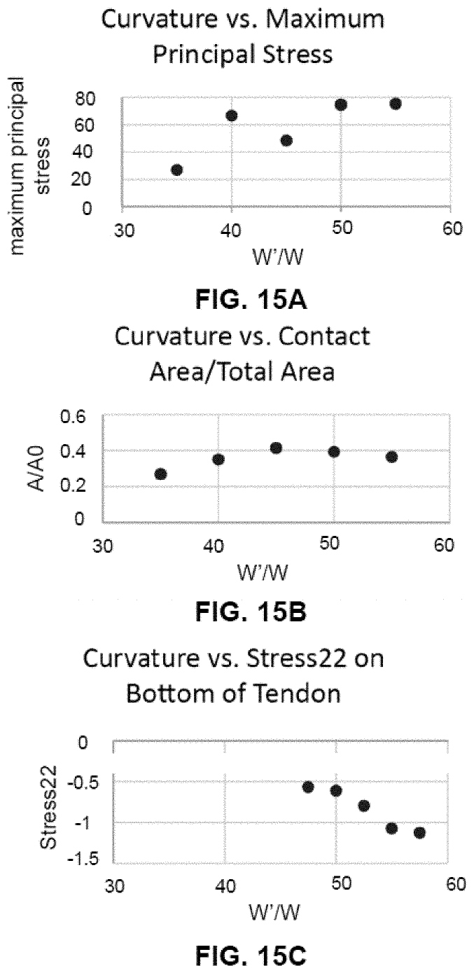

The same parameters were measured for the 3D model: The max principal stress, the area of the tendon in contact with the tooth after deformation, and the stress in the downward direction on the bottom of the tendon are illustrated in FIG. 15A, FIG. 15B, and FIG. 15C.

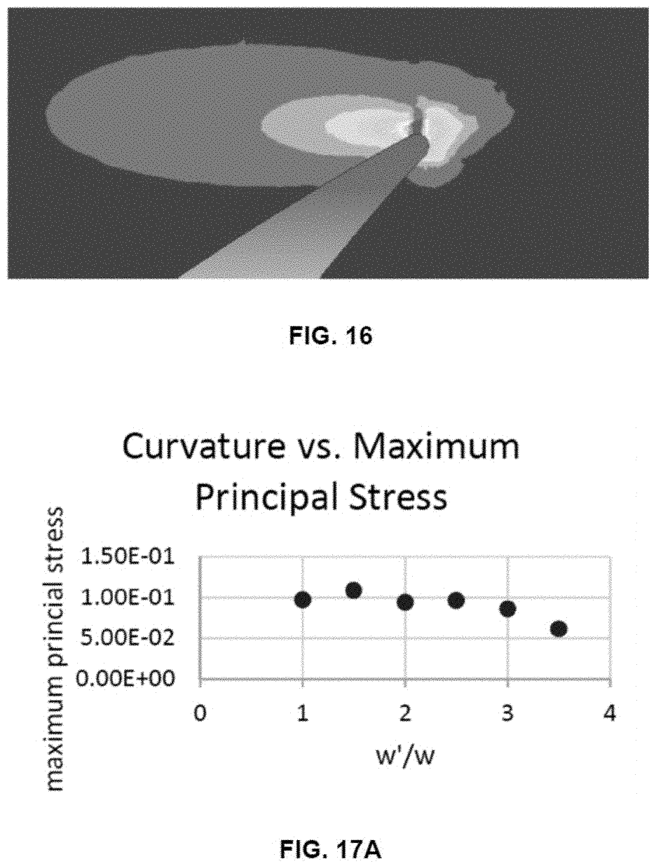

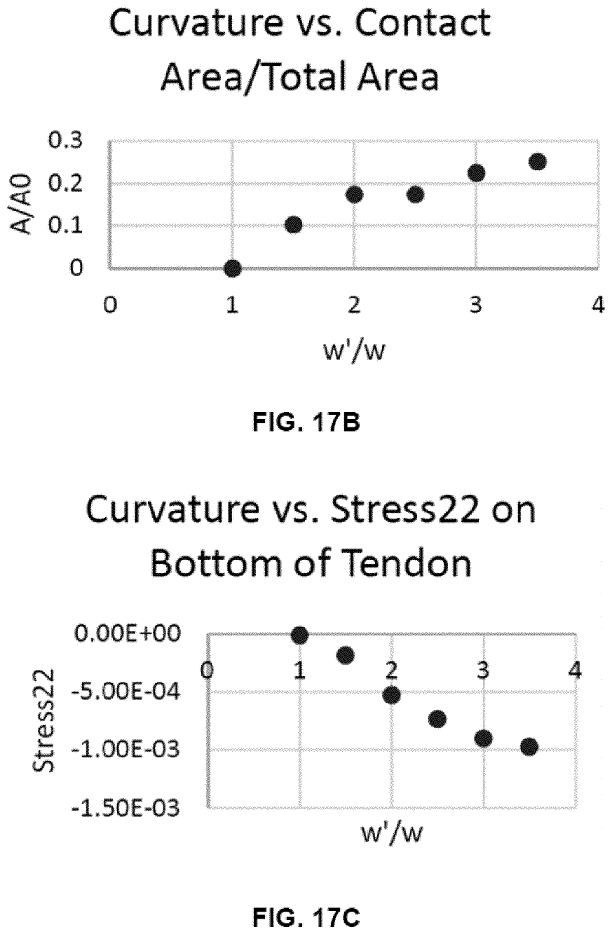

Orthotropic properties were added to the 2D circle model tendon to more accurately model the tendon. Since the stress was applied as a displacement, the maximum principal stress and vertical stress on the bottom of the tendon may not be normalized. FIG. 16 is a contour map showing orthotropic stress distribution for the 2D circle model with w′/w=2. FIG. 17A, FIG. 17B, and FIG. 17C are graphs showing the max principal stress, area, and stress on the bottom of the tendon, respectively, in the 2D orthotropic model.

The tendon-to-bone insertion transfers loads from a relatively compliant tendon to a relatively stiff bone. Once this insertion has been torn, it can be hard to redistribute the stress in a way that allows the tendon to heal effectively. Attaching an array of hooks to the bone to hold on to the tendon can help the tendon to heal.

Several models were built in this experiment to figure out the best dimensions for the hooks: 1) a 2D model built using the spline feature in Abaqus, 2) a 2D model using two circles for the inside and outside of the tooth, 3) a 3D model using a cone as the tooth, and 4) a model adding orthotropic properties to the 2D circle model.

The results from the spline model clearly show a point where the curvature is optimal. The goal was to find an optimum point where the contact stress and area may be maximized without an excessive jump in maximum principal stress. The area plot plateaus at w′/w=2.5 while the stress plateaus around w′/w=3, so the ideal curvature with this design should be around w′/w=2.5. The maximum stress is also increasing at this point. The plotted models for the two circles model plateaued at the same curvature for the contact area and stress on the bottom face of the tendon. The maximum principal stress values were similar for both models, but the circle model had higher contact stress indicating that this design may be better. The slight fluctuation in the maximum principal stress is most likely due to the model mesh not being super fine and due to the contact interaction.

The 3D model shows the same basic trends as the 2D model. The max principal stress increases and seems to peak at 50°. The w′/w is around 2 for all the 3D models. However, the base of each 3D model increases as the angle increases. As the cone was tilted farther forward, the base of the tooth became more elliptical and longer and the area taken up by the base of the cone increased. This makes the 3D model hard to compare to the 2D models. Qualitatively the 3D model's plot of stress on the bottom of the tendon seems to plateau at around the same time as the 2D model. The area of the tendon in contact with the bone has a slight parabolic function to it. This is mostly due to the area on the bottom of the tendon decreasing as the area is taken up by a larger cross-section of the cone.

The orthotropic model was the same geometrically as the circle model, but the values were not the same. The plots did not seem to plateau in the curvature that was tested. The plots for contact stress and area seem to flatten out slightly, but not plateau as the plots for the models with isotropic properties. This may be due to the tendon being softer and thus being more manipulated by the tooth at higher curvatures. This means that the optimal curvature for a more accurately parameterized tendon may be higher than the isotropic models show. The maximum principal stress also decreases for the higher curvature models suggesting that a higher curvature may be better. There were some struggles with getting the orthotropic model to converge since the tendon was so soft compared to the bone and the point of the tooth created high stress and deformation concentrations. A compromise between the level of the orthotropic properties (the young's modulus and shear modulus were raised from the values found in previous papers and the displacement on the tendon was decreased by a factor of 10) was reached. This compromise allowed the models to converge while still showing the general trends of the orthotropic properties in the tendon.

The 2D isotropic models suggested that increased slope or curvature of teeth increased contact stress and contact area at the expense of mild increases in stress concentration. Although these stress concentrations are large and might lead to local tearing of the tissue, the stresses typically reach a plateau. The plateaus reached in contact stress and contact area thus suggest an optimum at which performance is maximized. Plateaus in these parameters were not reached over the range of parameters studied in the 3D simulations or the orthotropic tendon.

### Example 3

### Finite Element Models of Various Tooth Shapes

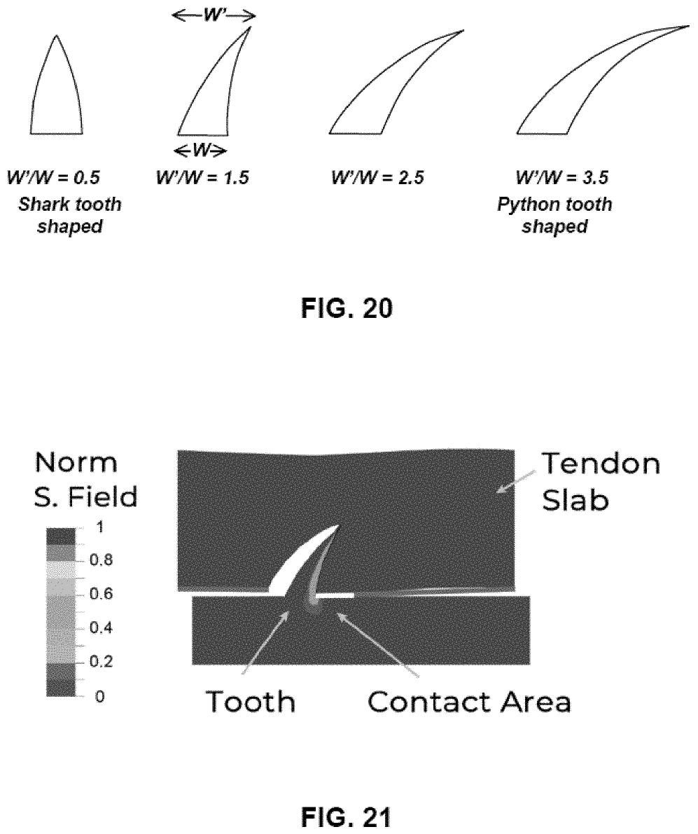

Rotator cuff tears are common, affecting more than 50% of patients over the age of 65 and resulting in pain and loss of shoulder function. Rotator cuff surgical repair is one of the most common shoulder procedures performed clinically. Unfortunately, healing after rotator cuff repair is a well-known clinical challenge, with reported failure rates as high as 94%. In the current standard double-row suture bridge repair, the suture punctures through bone and tendon at only two anchor points, thus leading to high concentrated forces (stresses) being transferred from tendon to bone in shear at these two points (FIG. 18). In the current study, a new approach for tendon-to-bone repair inspired by the remarkable grasping ability of python teeth on their prey is proposed, which distributes the stress over the entire area of a tooth array (FIG. 19). Unlike triangular shark teeth that function to cut their prey, python teeth have a curvature that grasps prey, prevents escape, and enables ingestion of entire, intact animals. To test the hypothesis that tooth shape drives the balance between cutting and grasping, finite element models and experiments were performed to quantify the interaction between tendons and seven different tooth designs, ranging from shark-like to python-like (FIG. 20).

Finite element models (FEM) of simple 2D and 3D models of frictionless teeth in a homogeneous “tendon” were studied using Abaqus (Dassault Systems). Images and microCT scans were used to determine the radii of Python molurus teeth. Seven different tooth geometries were examined in 2D, as defined by the dimensionless ratio w′/w (FIG. 20), which ranged from 0.5 to 3.5. A hard contact interaction was applied in the normal direction between the tooth and the tissue surfaces. The load was applied as a displacement of the tendon boundaries; periodic boundary conditions were applied to model an infinite row of teeth. The mesh was refined at the (rounded) tip of the tooth until convergence was reached. The maximum principal stress concentration, the contact area beneath the tooth (FIG. 2A), and the average tissue/gum contact pressure were estimated.

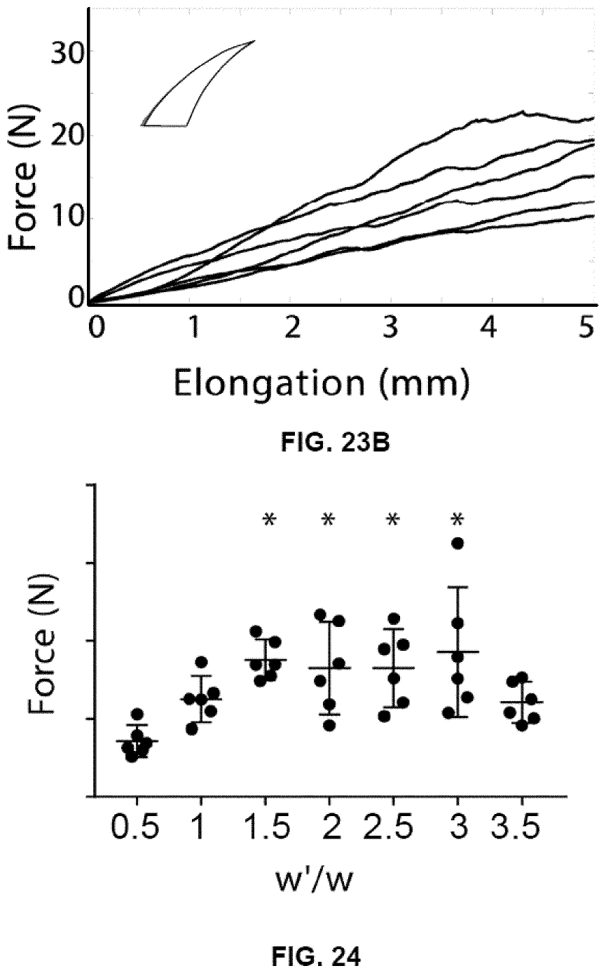

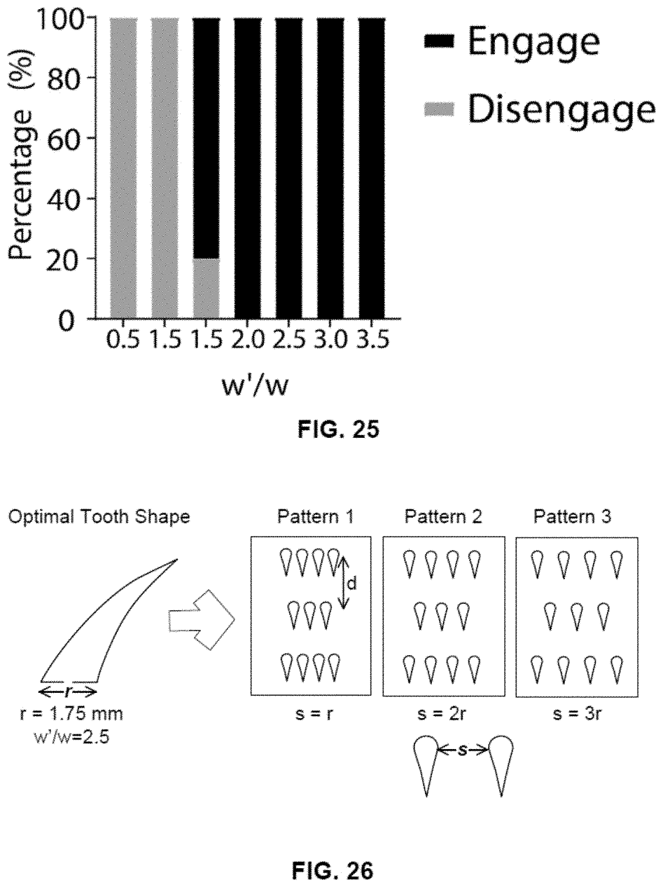

A modified single lap shear test was developed to test the grasping capacity of a tooth in a bovine deep digital flexor tendon (3.9″×2.4″×0.125″, N=14). Each fixture contained one 3D printed tooth (EDEN 260VS, Stratasys LTD) made with VeroWhitePlus™, a rigid and durable material with a modulus of elasticity of 2500 MPa. Each 3D printed tooth shape (N=6) was inserted into the precut tendon block so that the entire tooth was fully within the tendon. The uniaxial tension test was then performed at 0.05 mm/sec for up to 10 mm of displacement (TA instruments, ElectroForce). From the force elongation curves (FIG. 23A and FIG. 23B), peak force for 5 mm elongation (FIG. 24), stiffness, and energy to yield were determined. Tooth engagement with the tendon (FIG. 25) was determined by visual inspection and verified through video captured during testing. Groups were compared using ANOVA with a threshold for statistical significance defined at p<0.05.

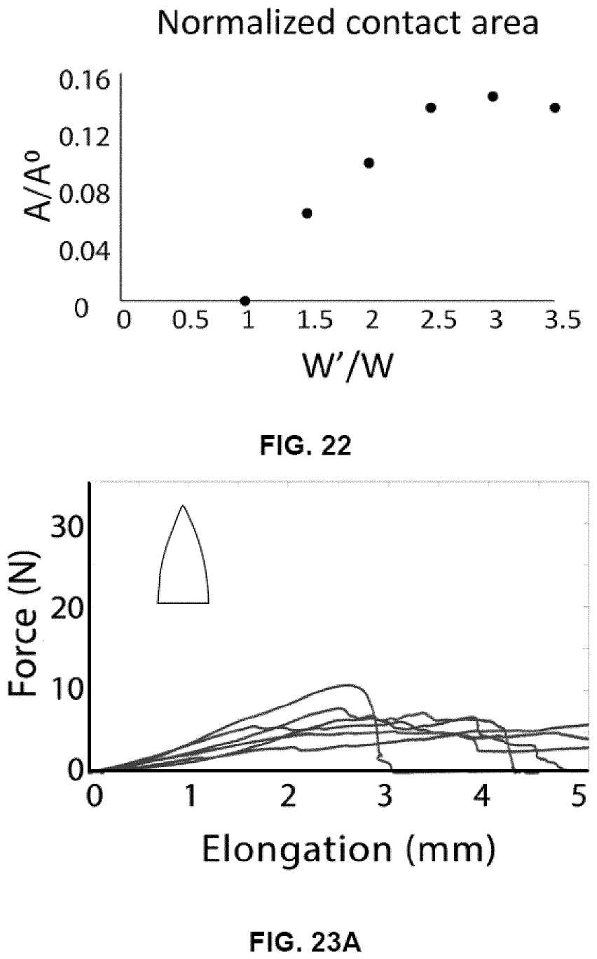

FEM results showed that the contact area between the tooth and the tendon increased with increasing w′/w, consistent with the hypothesis that python-shaped teeth are designed for grasping (FIG. 22). The maximum principal stress concentration remained constant for tooth shapes of w′/w=1, 1.5, 2, and 2.5 and decreased for w′/w=3 and 3.5. There was a significant increase in maximum force with increasing w′/w (FIG. 24, * p<0.05, when compared to w′/w=0.5). There was a dramatic decrease in the number of teeth that completely disengaged with increasing w′/w (FIG. 25). There was no significant difference between groups for stiffness and energy to yield.

FEM and experimental results revealed a strong non-linear relationship between tooth curvature and tendon grasping, implying an optimal tooth shape for the design of a tooth array device for tendon-to-bone repair. An optimal tooth shape maximizes the contact area without increasing the maximum principal stress concentration. From the modeling results, a shape with w′/w=2.5 was optimal, as the contact area did not increase with further increases in curvature. This shape is supported by experimental results, where the maximum force through 5 mm of displacement increased through w′/w=2.5. In addition, as evidenced by the force-displacement curves and the visual determination of tooth engagement, the tooth completely disengaged from the tendon at low values of w′/w. Ultimately, the device for enhanced tendon-to-bone repair will include an array of teeth, as shown in FIG. 19. The particular arrangement of these teeth may be further modified to ameliorate tissue-level shear lag inherent to tendon-to-bone repair.

Improving the fixation of tendon to bone during rotator cuff repair may impact healing and functional recovery. A bioinspired device to better grasp the tendon may reduce the high rupture rates currently observed after rotator cuff repair.

### Example 4

### Finite Element Models of Tooth Arrays

Rotator cuff tears are common, affecting more than 50% of patients over the age of 65 and resulting in pain and loss of shoulder function. Rotator cuff surgical repair is one of the most common shoulder procedures performed clinically. Unfortunately, healing after rotator cuff repair is a well-known clinical challenge, with reported failure rates as high as 94%. In the current standard double-row suture bridge repair, the suture punctures through bone and tendon at only two anchor points, thus transferring high concentrated forces from tendon to bone in shear at these two points (FIG. 19). Due to the high stress concentrations at the suture-tissue interfaces, the sutures can rupture or cut through the tendon, leading to failure of the repair.

In this study, a biomimetic approach to augment standard tendon-to-bone repair inspired by the remarkable grasping ability of python teeth on their prey is proposed. It is hypothesized that an array of teeth interposed between tendon and bone can enhance tendon-to-bone repair mechanics through better load distribution across the repair site (FIG. 19). A strong non-linear relationship between tooth curvature and tendon grasping was demonstrated in Example 3, implying an optimal tooth shape for grasping soft tissue. In this experiment, the single tooth optimization is extended to a clinically relevant tooth array device. Finite element models and experiments were performed to optimize spacing and size of tooth arrays for rotator cuff repair (FIG. 26). It was hypothesized that the grasping strength of the tooth array may be highest when stresses were evenly distributed among the teeth.

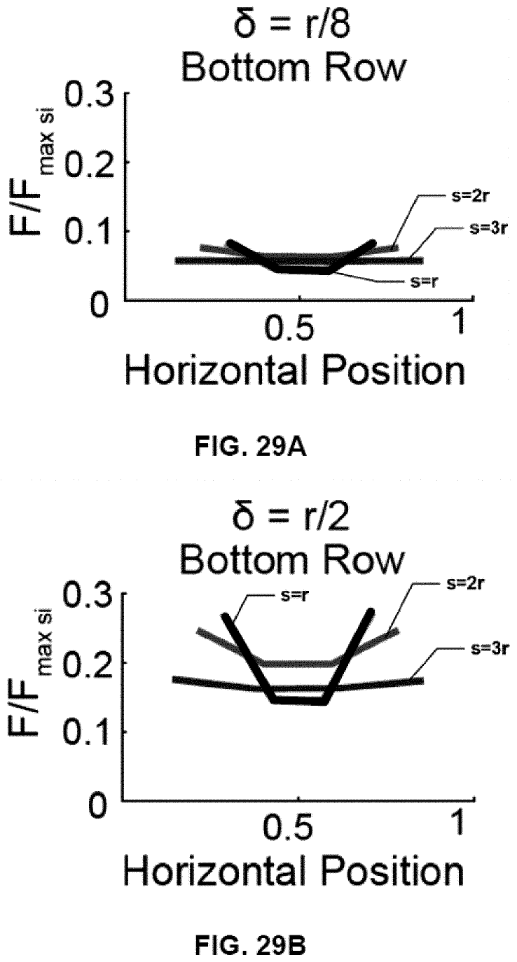

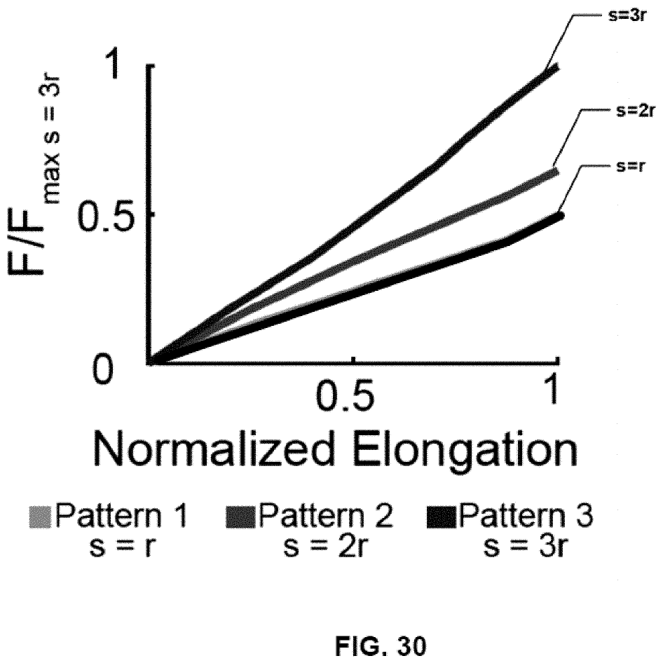

Finite element models (FEMs) of frictionless teeth (radius R) in a homogeneous, orthotropic “tendon” were studied using finite element analysis (Abaqus, Systemes, Waltham, Mass., USA). Three 2D tooth array patterns were examined, with the following spacing between adjacent teeth: (1) s=R (2) s=2R, and (3) s=3R (patterns 1, 2, and 3, respectively). Arranged in triangular arrays, in each pattern the teeth in the second row were placed equidistantly between the teeth in the first and third row (FIG. 26). Hard contact interaction was applied in the normal direction between the teeth and the tissue surfaces. A small gap was inserted into the rows closer to the displacement to account for how the tendon is placed on the teeth experimentally. As the tendon boundary was displaced uniformly, the total force on the tendon (FIG. 30) and distribution of force across teeth (FIG. 29) were recorded.

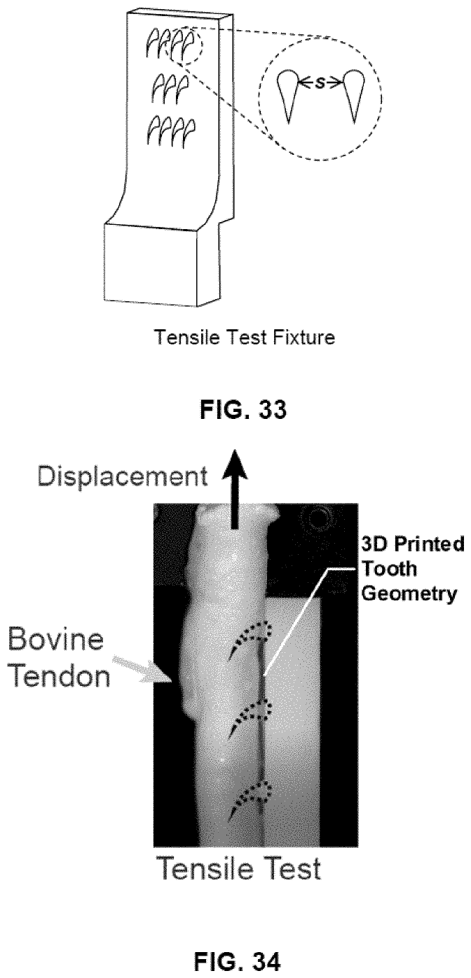

To assess shear lag, a modified single lap shear test was developed to test the grasping capacity of three different teeth array patterns in bovine deep digital flexor tendon (3.9″×2.4″×0.125″, N=8,9,10). Each fixture (FIG. 33) contained an array of 3D printed teeth (EDEN 260VS, Stratasys LTD) made with VeroWhitePlusTM, a rigid and durable plastic with a modulus of 2500 MPa. Each 3D printed tooth array pattern was inserted into the precut tendon block so that all teeth were fully engaged within the tendon, as illustrated in FIG. 34. The test was then performed at 0.05 mm/sec for up to 7 mm of displacement (TA instruments, ElectroForce). From the force-elongation curves, peak force for 7 mm elongation, yield, stiffness, and energy to 7 mm were determined. Tooth engagement with the tendon was determined by visual inspection and verified through video captured during testing. Groups were compared using ANOVA with a threshold for statistical significance defined at p<0.05.

To translate the idealized model and shear lag test results for clinical tendon-to-bone repair, a rotator cuff-specific device was designed using SolidWorks (Dassault Systèmes, Waltham, Mass., USA). The device illustrated in FIG. 27 consisted of the optimized array of teeth placed in a curved, flexible base that matched the curvature and dimensions of the humeral head supraspinatus tendon attachment site (0.67″×0.4″ footprint area). The fit of the device at the repair site and the qualitative grasping ability of the teeth were evaluated in one human cadaver shoulder for 3 different device prototypes. To check for the best design fit at the repair site, the following criteria were considered: (i) The device surface should match the attachment site surface, (ii) the device should not encroach on the articular cartilage, and (iii) the base thickness should be no more than 2 mm.

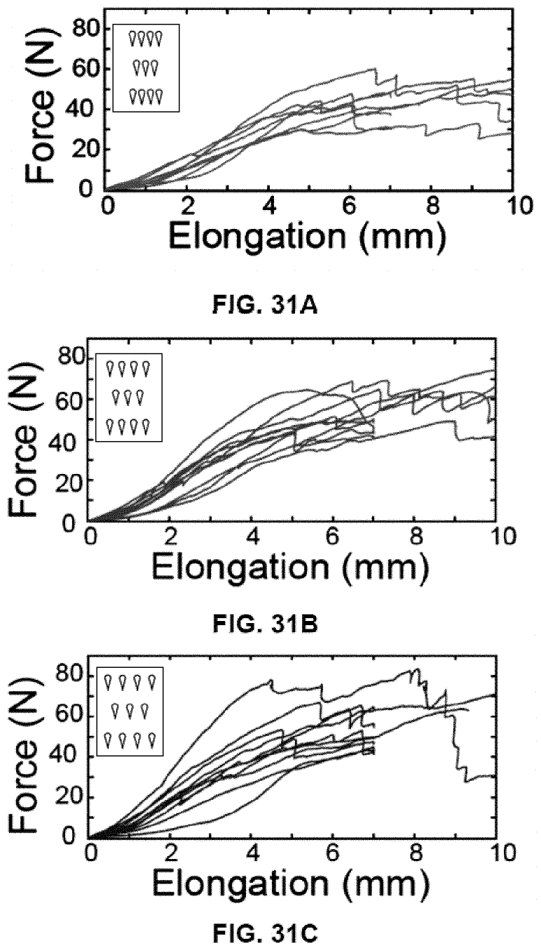

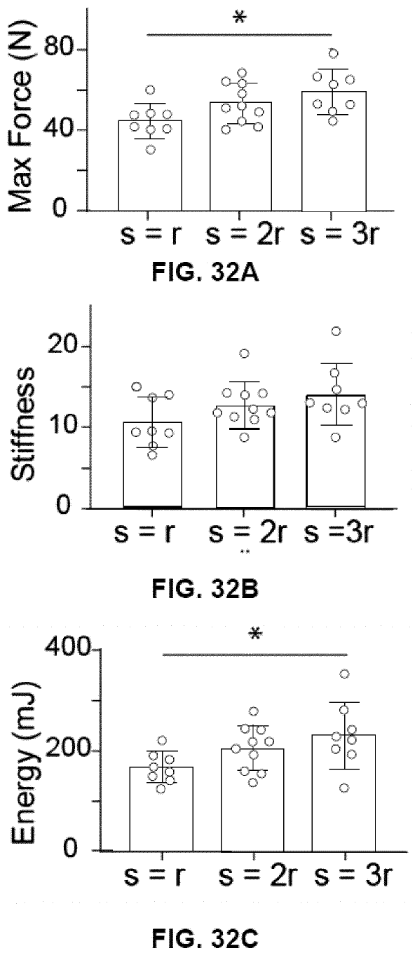

FEM results showed that wider spacing led to a uniform distribution of force amongst teeth, indicating that Pattern 3 (s=3r) may have the highest tearing strength (FIG. 29). Supported by experimental results, shear lag tests through 7 mm of displacement demonstrated maximum forces of 44.9 N±8.5 N (n=8), 53.8 N±9.9 N (n=10), and 59.3 N±11 N (n=8) for tooth patterns 1, 2 and 3, respectively (FIG. 31A, FIG. 31B, and FIG. 31C). Consistent with this, there was a significant increase in maximum force and energy with increasing horizontal tooth spacing (FIG. 32A, FIG. 32B, and FIG. 32C, * p<0.05, when compared to Pattern 1 with s=r). All force-displacement curves began concave-up, and there were no significant differences between groups for stiffness (FIG. 32B). Based on the above results, a device configuration (FIG. 27) interposed between tendon and bone at the repair site was chosen that fit the curved attachment footprint region and did not trespass the articular cartilage.

FEM and experimental results revealed a strong relationship between tooth spacing and tendon grasping, implying an optimal design space for a grasping device to augment tendon-to-bone repair. A python-tooth-inspired device may enhance tendon-to-bone repair by better distributing loads across the repair site. Modeling revealed that increasing tooth spacing evens out loads over each row of teeth, explaining the strengthening of the repair observed in Pattern 3 (s=3r). The concave-up character of the experimental force-displacement curves was consistent with the proximal rows of teeth engaging prior to distal rows. Combined with current repair techniques, a grasping device using Pattern 3 (s=3r) may improve repair strength by over 25% (˜78N), thus substantially reducing the risk of repair site failure and improving healing.

Cadaver tests showed that the tooth array configuration illustrated in FIG. 27 (far right) fit well at the repair site interposed between the tendon and bone. The device was seated in the native cavity between the greater tuberosity and the articular cartilage of the humerus with the teeth rising to grasp the tendon.

Improving the tendon-to-bone fixation during rotator cuff repair may improve outcomes after rotator cuff repair by reducing the high rupture rates currently observed after rotator cuff repair.

### Example 5

### Orthopedic Fixation Device with Porous Base

Repair of torn tendon-to-bone and ligament-to-bone attachments is a perennial challenge in orthopedic surgery. Some soft tissue repairs have very high failure rates. For example, high anatomical failure rates are associated with rotator cuff repairs. For example, 20-94% of rotator cuff repairs result in the recurrence of tears. Multiple factors affect rotator cuff healing, including patient-related factors, as well as surgical factors. Increased strength of the initial repair is thought to improve healing rates. However current methods of rotator cuff repair utilizing multiple sutures are accompanied by concerns about strangulating tissue and impeding blood supply to the tissues.

The orthopedic fixation device described in the example may be used to provide effective fixation and compression during post-surgical healing while offloading high stresses and allowing neovascularization. The device, shown illustrated in FIG. 35, is potentially applicable to many different anatomical sites in orthopedics, as well as other surgical subspecialties.

Current technology for this surgery involves suturing the tendon to bone. Suture techniques potentially allow for gap formation at the interface, another factor associated with poor healing. Poor soft tissue-hard tissue fixation after surgical repair remains a clinical challenge since a strong and tough attachment is not currently available using existing approaches. An advantage of the device of this experiment is that it provides more uniform contact between tendon and bone by augmenting or replacing sutures with a field of attachment points.

The device illustrated in FIG. 35 is inspired by the recurved teeth of pythons, designed to keep prey in the python's grasp as the prey succumbs to constriction. Inspired by this natural grasping method, the device includes an array of tines secured to a biomaterial base. The base interfaces directly with a hard or soft tissue surface such as tendon and bone. The array of tines achieves fixation by means of a recurved design, which favors unidirectional traction of the tissue towards the base, thereby achieving a stable and robust grip. The disclosed device can be implanted using current double-row repair methods, as illustrated in FIG. 36. The base is fixed to the hard tissue using suture anchors similar to those used for existing double row repair methods, and the soft tissue is gripped by the tines, as illustrated in FIG. 36. The device is placed between the soft tissue and bone, forming a strong tensile attachment.

The base of the device is porous. The lattice structure of the base may be created using the software nTopology Element in some aspects. The lattice shape is hexagonal and is only applied at the base, and not on the teeth or close to the threads for the sutures.

Referring again to FIG. 35, the base 104 may have a device length 3502 ranging from about 30 mm to about 34 mm and a device width ranging from about 12 mm to about 14 mm. The tine array 102 may have an array length 3508 of about 15 mm and an array width 3506 of about 7.5 mm. In various aspects, the dimensions of the device vary depending on individual patient morphology. In one aspect, the device 100b has a base with a device length 3502 of about 32 mm, a device width of about 13 mm, an array length 3508 of about 15 mm, and an array width 3506 of about 7.5 mm.

### Example 6

### Orthopedic Fixation Using Tooth Array Device

To demonstrate a repair of a rotator cuff using the tooth array device with four fastener holes described above and illustrated in FIGS. 40 and 41, the following experiments were conducted.

The disclosed device 100c was implanted alongside current double-row repair methods as illustrated in FIG. 42. The fixation into bone was accomplished through 4 suture holes 116a, 116b, 116c, and 116d, placed in the 4 corners of the rectangular base. The medial side of the device 100c was fixed into bone 210 by loading each axillary suture 4202a and 4202b from the suture anchors 206a and 206b, respectively, through the respective medial suture holes 116a and 116c and tying them using surgical slip tie knots 4204a and 4204b. The lateral side of the device 100c was fixed into bone 210 by first loading additional sutures 4206a and 4206b from the suture anchors 206a and 206b, respectively into the lateral suture holes 116b and 116d, followed by loading these sutures into the suture anchors 206a and 206b to fixate into bone 210.

Double row repairs with suture anchors were used to repair 4 paired cadaver shoulders with and without the device as described above. The use of the device with the double row repair led to improvements of 28%, 43%, 62%, and 139% in strength relative to the corresponding repair without the device (average strength improvement of 68%).

An additional fixation repair was performed using the device 100c as illustrated in FIGS. 43A, 43B, 43C, and 43D. As illustrated in FIG. 43A, medial sutures 4302a and 4302c were loaded into medial suture anchors 4304a and 4304c and the anchors 4304a and 4304c were inserted into the underlying bone 4310 at least partially within the desired attachment footprint 4308. Referring to FIG. 43B, the device 100c was positioned within the desired attachment footprint 4308, medial sutures 4304a and 4304c were loaded through suture holes 116a and 116c, and the sutures 4304a and 4304c were secured with knots 4312a and 4312c to achieve medial fixation. Referring to FIG. 43C, lateral sutures 4302b and 4302d were loaded into suture holes 116b and 116d. In some aspects, the medial sutures may be preloaded before positioning the device within the device footprint, or the medial sutures may be loaded in situ using preloaded suture loaders as described herein and illustrated in FIGS. 44A and 44B. Referring to FIG. 43D, lateral sutures 4302b and 4302d were then loaded into lateral anchors 4304b and 4304d, which were already inserted into the underlying bone 4310. Lateral sutures 4304b and 4304d were secured to lateral anchors 4304b and 4304d using knots 4312b and 4312d to complete lateral fixation. In some aspects, the lateral sutures may be preloaded into the lateral suture anchors, and the loaded through the corresponding lateral suture holes in situ, optionally using preloaded suture loaders as described herein and illustrated in FIGS. 44A and 44B.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

1. A device for joining a first tissue to a second tissue in a patient in need, the device comprising:
a. a base comprising opposed first and second surfaces, the base defining four suture holes extending through the first and second surfaces of the base, each suture hole configured to receive at least one suture passing between the first and second surfaces; and
b. a plurality of recurved tines oriented to a tine axis and extending from the first surface of the base, the plurality of recurved tines providing unidirectional traction of the first tissue along the tine axis toward the first surface;
wherein the first tissue is secured to the first surface of the device at the plurality of recurved tines and the second tissue is secured to the device at the second surface to join the first tissue to the second tissue.

2. The device of claim 1, wherein the at least one suture is configured to secure to at least one suture anchor, the at least one suture anchor secured to the second tissue.

3. The device of claim 2, wherein at least a portion of the suture holes of the base are configured to receive anchor screws, and the device is secured to the second tissue using a combination of anchor screws and sutures.

4. The device of claim 1, wherein the first tissue and second tissue are selected independently from a hard tissue and a soft tissue.

5. The device of claim 3, wherein the hard tissue comprises a bone tissue.

6. The device of claim 3, wherein the soft tissue is a soft connective tissue selected from a ligament, a tendon, a muscle, a cartilage tissue, a meniscus, a fibrocartilage tissue, an adipose tissue, and any combination thereof

7. The device of claim 1, wherein the first tine axis is oriented in a direction opposite to the second tine axis.

8. The device of claim 1, wherein the base comprises essentially planar first and second surfaces.

9. The device of claim 1, wherein the base further comprises a flexible material wherein the base is further configured to conform to match a contour of the second tissue, to fold or roll to fit through an arthroscopic cannula, and any combination thereof.

10. The device of claim 1, wherein the base further defines a plurality of pores, each pore of the plurality of pores extending at least partly from the first surface to the second surface.

11. The device of claim 10, wherein at least a portion of the plurality of pores are arranged in a distribution pattern over at least a portion of the base, the pattern selected from one of a random distribution, a uniform distribution, a lattice distribution, and any combination thereof.

12. The device of claim 11, wherein the lattice distribution is selected from one of a row and column distribution, a cubic lattice distribution, and a hexagonal lattice distribution.

13. The device of claim 12, wherein the distribution pattern of the pores is configured to reduce or equalize stresses in the plurality of recurved tines, to equalize mechanical loadings in the plurality of recurved tines, to reduce maximum stress in the base, reduce a likelihood of failure of the base, and any combination thereof.

14. The device of claim 1, wherein at least one of the first surface and the second surface are contoured, the first surface contoured to conform to a surface of the first tissue, and the second surface contoured to conform to a surface of the second tissue.

15. The device of claim 1, wherein at least one of the first surface and the second surface are contoured, the first surface contoured to conform to a surface of the first tissue of an individual patient, and the second surface contoured to conform to a surface of the second tissue of the individual patient.

16. The device of claim 1, wherein the base further comprises a length ranging from about 5 mm to about 18 mm and a width ranging from about 3 mm to about 9 mm.

17. The device of claim 1, wherein the base comprises a length of about 17.5 mm and a width of about 8 mm.

18. The device of claim 1, wherein:
a. a portion of the plurality of recurved tines vary in a tine characteristic, the tine characteristic comprising a tine size, a tine spacing, a tine orientation different from the tine axis, or any combination thereof; and
b. each tine characteristic of each tine from the portion of the plurality of recurved tines is selected to reduce or equalize stresses in the tines, reduce stresses in the attached first tissue, and any combination thereof.

19. The device of claim 1, wherein the base comprises at least two base modules, wherein the at least two base modules are configured for insertion through an arthroscopic cannula and are further configured for assembly into the base after insertion.

20. The device of claim 19, wherein the at least two base modules are individually affixed to the second tissue using at least one suture, at least one anchor screw, or any combination thereof.
